(19) United States
(12) Patent Application Publication
(10) Pub. No.: US 2023/0390215 A1
(43) Pub. Date: Dec. 7, 2023

(54) SYSTEMS, DEVICES, AND FORMULATIONS FOR TIME-PHASED HISTAMINE DELIVERY

(71) Applicant: AgoneX Biopharmaceuticals, Inc., Santa Monica, CA (US)

(72) Inventors: Cristian Arnou, Santa Monica, CA (US); Neil Flanzraich, Santa Monica, CA (US)

(21) Appl. No.: 18/451,361

(22) Filed: Aug. 17, 2023

Related U.S. Application Data
(63) Continuation of application No. PCT/US2022/016786, filed on Feb. 17, 2022.
(60) Provisional application No. 63/150,785, filed on Feb. 18, 2021.
(60) Provisional application No. 63/150,783, filed on Feb. 18, 2021.

(51) Int. Cl. A61K 9/70; A61K 31/417; A61K 47/10; A61K 47/12; A61K 47/32; A61K 47/34

(52) U.S. Cl. CPC A61K 9/7084; A61K 31/417; A61K 47/10; A61K 47/12; A61K 47/32; A61K 47/34

(57) ABSTRACT

Aspects of the disclosure provided herein relate to compositions and methods for providing a drug delivery through a transdermal patch. Some other embodiments relate to a transdermal drug delivery patch comprising a bottom layer comprising a skin side liner, a middle layer comprising a gel, a top layer comprising a release liner, wherein the adhesive or gel comprises at least one drug capable of modulating histamine in a subject. Some other embodiments provided herein relate to a transdermal drug delivery system for delivery of at least one drug into a tissue membrane of a subject. Some other embodiments relate to a transdermal drug delivery patch comprising a bottom layer comprising a skin side liner, a middle layer comprising a gel/adhesive, a top layer comprising a release liner, wherein the gel comprises at least one drug capable of modulating histamine receptors in a subject.

### INCORPORATION BY REFERENCE TO RELATED APPLICATIONS

This application is a continuation of PCT/US2022/016786 filed, Feb. 17, 2022, which claims benefit of U.S. Provisional Application Nos. 63/150785 filed, Feb. 18, 2021 and 63/150,783 filed, Feb. 18, 2021. The aforementioned application is incorporated by reference herein in its entirety; and is hereby expressly made a part of this specification.

### FIELD

The present disclosure relates to compositions, devices, and methods for transdermal drug delivery, particularly transdermal histamine formulations and methods for administering the transdermal histamine to subjects by transdermal devices. In addition, systems for transdermal time-phased delivery of histamine are disclosed.

### DESCRIPTION OF THE RELATED ART

The viable epidermis is a region for drug binding, metabolism, active transport, and surveillance. In addition to keratinocytes, it contains melanocytes (dendritic cells found on the basement membrane and in the basal layer), Merkel cells (functioning as mechanoreceptors involved in the mediation of touch responses, found in the basal region), and Langerhans cells (dendritic cells playing a key role in protective immune function, present mainly in the stratum spinosum).

Histamine receptors are expressed in a number of resident and non-resident cells of the skin. The trans-dermal device is used for accessing the histamine receptors (H1R-H4R) expressed on the various skin cells. The similarity of receptors, the cellular ability to produce and respond to the same chemicals—histamine in particular—and the shared chemical-electric “language” of communication explain the simultaneous engagement of nerve cells and immuno-competent cells in allergic processes. No matter what triggers these reactions, and what cells get activated first, the reactions are immunologic, since major immune cells and their products take part in them. Eosinophils express various cell surface markers, such as Fc receptors, CCR3, PAF receptors, CD48, 2B4, Siglec-F and histamine receptors, which are responsible for cell-cell communication via various pathways.

### SUMMARY OF THE DISCLOSURE

Some embodiments relate to a patch for delivering an active agent to a subject in need thereof. In some embodiments, the patch comprises a top layer comprising an adhesive, a middle layer comprising an active agent, wherein the active agent is a histamine or salt thereof; and a bottom layer, wherein the bottom layer comprises a release liner. In some embodiments, the active agent is 0.001 μg/mL to about 30 μg/mL histamine or salt thereof. In some embodiments, the middle layer further comprises a carrier. In some embodiments, the carrier comprises water, saline, saccharides, polysaccharides, buffers, excipients, biodegradable polymers, liposomes, stabilizer, skin irritation reducer, and mixtures thereof. In some embodiments, the middle layer is a gel. In some embodiments, the gel is a hydrogel. In some embodiments, the release liner comprises a polyester liner or a polyethylene liner. In some embodiments, the top liner comprises a polyester liner or a polyethylene liner. In some embodiments, the patch is at least 1 cm by 1 cm. In some embodiments, the patch is at least 0.002 mm thick to about 2 mm thick. In some embodiments, the middle layer further comprises a permeation enhancing agent. In some embodiments, the middle layer further comprises an absorption enhancer. In some embodiments, the patch has a pH from about 1.5 to about 8.

Some embodiments relate to a method of modulating histamine in a subject. In some embodiments, the method comprises administering to the subject the patch according to an embodiment described herein. In some embodiments, the subject's plasma histamine levels are lowered. In some embodiments, the subject's blood histamine concentration is lowered. In some embodiments, the serum histamine level in the subject are lowered by at least 10 ng/ml. In some embodiments, the plasma histamine levels in the subject are lowered by at least 10 ng/ml. In some embodiments, the buffy coat histamine levels in the subject are lowered by at least 10 ng/ml. In some embodiments, the serum histamine levels in the subject are reduced from about 100 ng/ml to about 45 ng/ml. In some embodiments, the patch is applied to the subject's skin from about 0.5 hours to about 72 hours. In some embodiments, more than one patch is applied to the subject's skin. In some embodiments, more than one patch containing different concentrations of the active ingredient (histamine receptor modulator) is applied to the subject's skin. In some embodiments, a subsequent patch is applied to a subject's skin after 0.5 to about 72 hours after the first patch is applied to the subject's skin. In some embodiments, one or more patch is applied to the subject's skin over a 14 day treatment cycle.

Some embodiments relate to a method of ameliorating, reducing, or treating a migraine headache of a subject. In some embodiments, the method comprises administering to the subject a patch according to an embodiment as described herein. In some embodiments, the subject's plasma histamine levels are lowered. In some embodiments, the subject's blood histamine concentration is lowered. In some embodiments, the serum histamine level in the subject is lowered by at least 10 ng/ml. In some embodiments, the plasma histamine levels in the subject are lowered by at least 10 ng/ml. In some embodiments, the buffy coat histamine levels in the subject are lowered by at least 10 ng/ml. In some embodiments, the serum histamine levels in the subject are reduced from about 100 ng/ml to about 45 ng/ml. In some embodiments, the patch is applied to the subjects skin from about 0.5 hours to about 72 hours. In some embodiments, the more than one patch is applied to the subject's skin. In some embodiments, a subsequent patch is applied to a subject's skin after 0.5 to about 72 hours after the first patch is applied to the subject's skin. In some embodiments, one or more patch is applied to the subject's skin over a 14 day treatment cycle. In some embodiments, the histamine levels during a migraine event are lowered. In some embodiments, the histamine levels during a symptom free state is lowered.

Some embodiments relate to a method of modulating brain histamine levels in a subject with Parkinson's disease. In some embodiments, the method comprises administering to the subject a patch according to an embodiment as described herein. In some embodiments, the subject's plasma histamine levels are lowered. In some embodiments, the subject's blood histamine concentration is lowered. In some embodiments, the serum histamine level in the subject is lowered by at least 10 ng/ml. In some embodiments, the plasma histamine levels in the subject are lowered by at least 10 ng/ml. In some embodiments, the buffy coat histamine levels in the subject are lowered by at least 10 ng/ml. In some embodiments, the serum histamine levels in the subject are reduced from about 100 ng/ml to about 45 ng/ml. In some embodiments, the patch is applied to the subjects skin from about 0.5 hours to about 72 hours. In some embodiments, more than one patch is applied to the subject's skin. In some embodiments, a subsequent patch is applied to a subject's skin after 0.5 to about 72 hours after the first patch is applied to the subject's skin. In some embodiments, one or more patch is applied to the subject's skin over a 14 day treatment cycle up to 1 year treatment applied at varying time intervals.

Some embodiments relate to a method for modulating brain histamine levels in a subject with Alzheimer's disease. In some embodiments, administering to the subject the patch according to an embodiment as described herein. In some embodiments, the subject's plasma histamine levels are lowered. In some embodiments, the subject's blood histamine concentration is lowered. In some embodiments, the serum histamine level in the subject is lowered by at least 10 ng/ml. In some embodiments, the plasma histamine levels in the subject are lowered by at least 10 ng/ml. In some embodiments, the buffy coat histamine levels in the subject are lowered by at least 10 ng/ml. In some embodiments, the serum histamine levels in the subject are reduced from about 100 ng/ml to about 45 ng/ml. In some embodiments, the patch is applied to the subjects skin from about 0.5 hours to about 72 hours. In some embodiments, the more than one patch is applied to the subject's skin. In some embodiments, a subsequent patch is applied to a subject's skin after 0.5 to about 72 hours after the first patch is applied to the subject's skin. In some embodiments, the one or more patch is applied to the subject's skin over a 14 day treatment cycle. up to 1 year treatment applied at varying time intervals.

Some embodiments relate to a method of treating a subject with elevated histamine levels. In some embodiments, the method comprises administering to the subject a first dosing segment comprising the patch according to an embodiment as described herein and administering to the subject a second dosing segment comprising the patch according to an embodiment as described herein. In some embodiments, the method further comprises administering to the subject a third dosing segment comprising the patch according to an embodiment as described herein. In some embodiments, the method further comprises administering to the subject a fourth dosing segment comprising the patch according to an embodiment as described herein. In some embodiments, each patch from the first dosing segment and the second dosing segment comprises a different concentration of the active agent.

Some embodiments relate to a transdermal drug delivery system for delivering at least one drug into a tissue membrane of a subject. In some embodiments, the system comprises an applicator configured to supply a drug delivery patch, and a patch. In some embodiments, the patch comprises a top layer comprising an adhesive, a middle layer comprising at least one drug, and a bottom layer, wherein the bottom layer comprises a release liner, wherein the at least one drug is a histamine receptor modulator. In some embodiments, the histamine receptor modulator activates histamine H1 receptors. In some embodiments, the histamine receptor modulator activates histamine H2 receptors. In some embodiments, the histamine receptor modulator activates histamine H3 receptors. In some embodiments, the histamine receptor modulator activates histamine H4 receptors. In some embodiments, the histamine receptor modulator is histamine, a histamine salt, or a combination thereof. In some embodiments, the histamine salt is selected from the group consisting of histamine diphosphate, N-alpha methyl histamine, histamine phosphate, histamine dihydrochloride, or a combination thereof. In some embodiments, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof. In some embodiments, the histamine receptor modulator concentration is 0.001 μg/mL to about 100 μg/mL. In some embodiments, the middle layer further comprises a carrier. In some embodiments, the carrier comprises water, saline, saccharides, polysaccharides, buffers, excipients, biodegradable polymers, liposomes, stabilizer, skin irritation reducer, and mixtures thereof. In some embodiments, the middle layer is a gel. In some embodiments, the gel is a hydrogel. In some embodiments, the release liner comprises a polyester liner or a polyethylene liner. In some embodiments, the top liner comprises a polyester liner or a polyethylene liner. In some embodiments, the patch is at least 1 cm by 1 cm. In some embodiments, the patch is at least 0.002 mm thick to about 2 mm thick. In some embodiments, the middle layer further comprises a permeation-enhancing agent. In some embodiments, the middle layer further comprises an absorption enhancer. In some embodiments, the patch has a pH from about 3 to about 8. In some embodiments, the applicator is configured for microporation delivery of the patch. In some embodiments, the applicator is configured for microneedle delivery of the patch.

Some embodiments relate to a transdermal drug delivery system for delivering at least one drug into a tissue membrane of a subject. In some embodiments, the system comprises a patch. In some embodiments, the patch comprises a top layer comprising an adhesive, a middle layer comprising at least one drug, and a bottom layer, wherein the bottom layer comprises a release liner, wherein at least one drug is a histamine receptor modulator. In some embodiments, the histamine receptor modulator activates histamine H1 receptors. In some embodiments, the histamine receptor modulator activates histamine H2 receptors. In some embodiments, the histamine receptor modulator activates histamine H3 receptors. In some embodiments, the histamine receptor modulator activates histamine H4 receptors. In some embodiments, the histamine receptor modulator activates the histamine H1, H2, H3 and H4 receptors at various rates/levels over the application/exposure time. In some embodiments, the histamine receptor modulator is histamine, a histamine salt, or a combination thereof. In some embodiments, the histamine salt is selected from the group consisting of histamine diphosphate, N-alpha methyl histamine, histamine phosphate, histamine dihydrochloride, or a combination thereof. In some embodiments, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof. In some embodiments, the histamine receptor modulator concentration is 0.0001 μg/mL to about 100 μg/mL. In some embodiments, the middle layer further comprises a carrier. In some embodiments, the carrier comprises water, saline, saccharides, polysaccharides, buffers, excipients, biodegradable polymers, liposomes, stabilizer, skin irritation reducer, and mixtures thereof. In some embodiments, the middle layer is a gel. In some embodiments, the gel is a hydrogel. In some embodiments, the release liner comprises a polyester liner or a polyethylene liner. In some embodiments, the top liner comprises a polyester liner or a polyethylene liner. In some embodiments, the patch is at least 1 cm by 1 cm. In some embodiments, the patch is at least 0.2 mm thick to about 2 mm thick. In some embodiments, the middle layer further comprises a permeation-enhancing agent. In some embodiments, the middle layer further comprises an absorption enhancer. In some embodiments, the patch has a pH from about 3 to about 8. In some embodiments, the patch further comprises a rate controlling membrane. In some embodiments, the adhesive is a pressure-sensitive adhesive. In some embodiments, the top layer comprises a non-woven backing.

### BRIEF DESCRIPTION OF THE DRAWINGS

FIG. 1A is a photograph of the backing side of an embodiment of the transdermal patch; FIG. 1B is a photograph of a side angle of the backing side of an embodiment the transdermal patch; FIG. 1C is a photograph of one side of a pressure sensitive transdermal patch (Drug-in-Adhesive); FIG. 1D is a photograph of a side perspective of a pressure sensitive transdermal patch (Drug-in-Adhesive); FIG. 1E is a photograph of one side of a 2″×2″ pressure sensitive transdermal patch on the shoulder skin of a subject (Drug-in-Adhesive).

FIG. 2 is a photograph of the skin side of the transdermal patch.

FIG. 3 is a photograph of an embodiment of transdermal patch.

FIG. 4 depicts a transdermal delivery system according to one embodiment of the disclosure.

FIG. 5A illustrates a graph representing experimental results from a transdermal flux of histamine hydrochloride through porcine skin on a Franz cell at room temperature; FIG. 5B illustrates a graph representing experimental results from a transdermal flux of histamine hydrochloride through porcine skin on a Franz cell at room temperature.

FIG. 6A illustrates a graph representing experimental results correlating Franz cell receptor solution (at room temperature) histamine concentration and shoulder viable epidermis histamine concentration; FIG. 6B illustrates a graph representing experimental results correlating Franz cell receptor solution (at room temperature) histamine concentration and shoulder viable epidermis histamine concentration.

FIG. 7A illustrates a graph representing experimental results for histamine levels during symptom free states; FIG. 7B illustrates a graph representing experimental results for histamine levels during migraine events.

FIG. 8 illustrates a graph representing experimental results for a plasma histamine level of myelogenous leukemia patients versus healthy patients.

FIG. 9 illustrates a graph representing experimental results for a plasma histamine level of coronary disease patients versus healthy patients.

FIG. 10 illustrates a graph representing experimental results for a brain histamine level of brain histamine levels in patients with Parkinson's disease versus healthy patients.

FIG. 11 illustrates a graph representing experimental results for a brain histamine level of Alzheimer's Disease versus healthy patients.

FIG. 12 illustrates a graph representing an in vitro transdermal flux of histamine hydrochloride from a 2.7 μg/ml hydrogel patch.

FIG. 13 illustrates a graph representing an in vitro transdermal flux of histamine hydrochloride from a 30 μg/ml hydrogel patch.

FIG. 14 illustrates a line graph representing cumulative in vitro transdermal flux (ng/cm2) of Histamine Dihydrochloride through porcine skin from 5 μg/ml versus time (hours) at body temperature.

FIG. 15 illustrates a line graph representing cumulative in vitro transdermal flux (ng/cm2) of Histamine Dihydrochloride through porcine skin from 5 μg/ml versus time (hours) at body temperature.

FIG. 16 illustrates a computed algorithm predicted histamine concentration gradient derived from the InLine7 cell results as a function of skin thickness starting with the Epidermis and continuing with the Dermis and Hypodermis.

FIG. 17 illustrates a never and mast cell interaction where both contain histamine receptors (H1R-H4R) and the bidirectional interaction between nerve and mast cells.

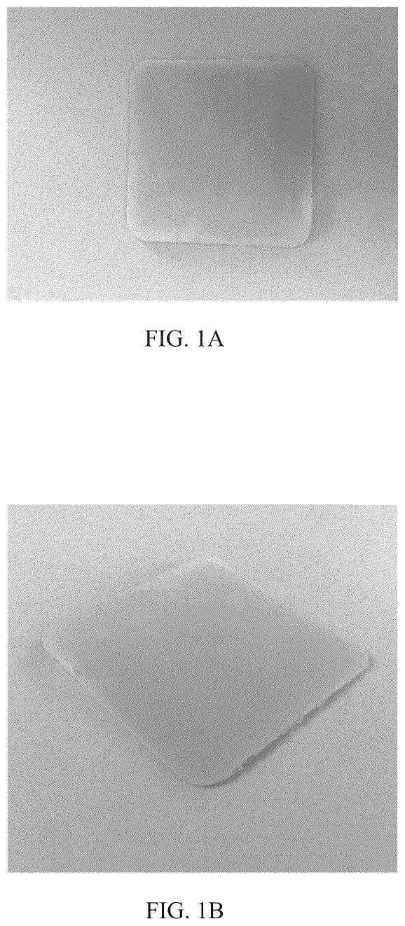

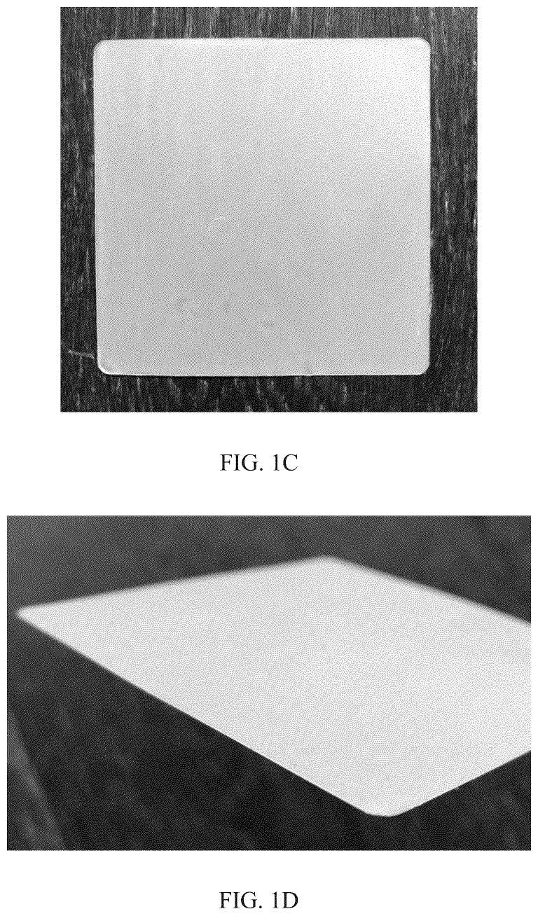

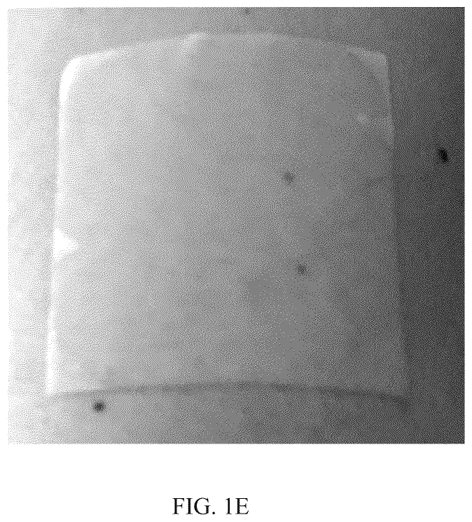

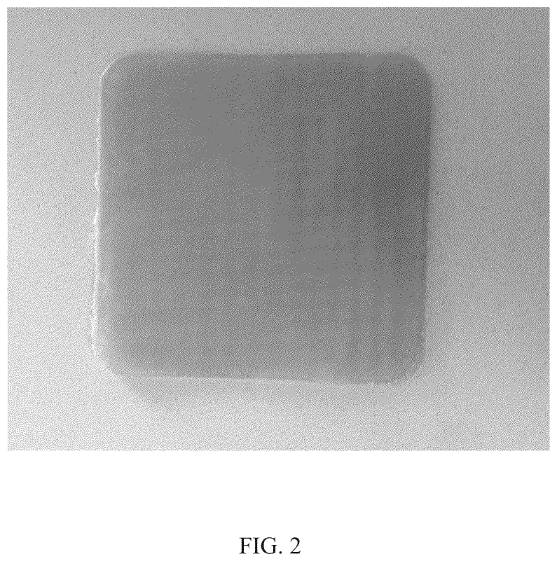

### DETAILED DESCRIPTION

The present disclosure can be understood more readily by reference to the following detailed description, examples, drawing, and claims, and their previous and following descriptions. However, before the present devices, systems, and/or methods are disclosed and described, it is to be understood that this invention is not limited to the specific devices, systems, and/or methods disclosed unless otherwise specified. It is also to be understood that the terminology used herein is for the purpose of describing particular aspects only and is not necessarily intended to be limiting.

This description is provided as an enabling teaching of the invention. To this end, those skilled in the relevant art will recognize and appreciate that many changes can be made to the various aspects of the invention described herein, while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present invention. Thus, this description is provided as illustrative of certain principles of the present invention and not in limitation thereof.

### Definitions

As used throughout, the singular forms “a,” “an,” and “the” include plural referents unless the context clearly dictates otherwise. Thus, for example, reference to “a filament” can include two or more such filaments unless the context indicates otherwise.

Ranges can be expressed herein as from “about” or “approximately” one particular value, and/or to “about” or “approximately” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. For example, “about” or “approximately” can mean within 1 or more than 1 standard deviations, per the practice in the art. Alternatively, “about” or “approximately” can mean a range of up to 20%, up to 10%, up to 5%, and up to 1% of a given value.

As used herein, the terms “optional” or “optionally” mean that the subsequently described event or circumstance may or may not occur, and that the description includes instances where said event or circumstance occurs and instances where it does not.

As used herein, “stratum corneum” refers to the outermost layer of the skin, consisting of from about 15 to about 20 layers of cells in various stages of drying out. The stratum corneum provides a barrier to the loss of water from inside the body to the external environment and from attack from the external environment to the interior of the body.

As used herein, “tissue” refers to an aggregate of cells of a particular kind, together with their intercellular substance, that forms a structural material. At least one surface of the tissue must be accessible to the device. The preferred tissue is the skin. Other tissues suitable for use with embodiments of this disclosure include mucosal tissue and soft organs.

As used herein, the term, “interstitial fluid” is the clear fluid that occupies the space between the cells in the body.

As used herein, the term “biological fluid” is defined as a fluid originating from a biological organism, including blood serum or whole blood as well as interstitial fluid.

The term “subject” includes animals (for example, mammals, for example, cats, dogs, horses, pigs, cows, sheep, rodents, rabbits, squirrels, camels, bears, primates (for example, chimpanzees, gorillas, and/or humans). It also includes transgenic animal models.

As used herein, a “tissue membrane” can be any one or more epidermal layers of a subject. For example, in one aspect, the tissue membrane is a skin layer that includes the outermost layer of the skin, i.e., the stratum corneum. In an alternative aspect, a skin layer can include one or more backing layers of the epidermis, commonly identified as stratum granulosum, stratum malpighii, and stratum germinativum layers. It will be appreciated by one of ordinary skill in the art that there is essentially little or no resistance to transport or to absorption of a permeant through the backing layers of the epidermis. Therefore, in one aspect, an at least one formed pathway in a skin layer of a subject is a pathway in the stratum corneum layer of a subject. Further, as used herein, “stratum corneum” refers to the outermost layer of the skin, typically containing from about 15 to about 20 layers of cells in various stages of drying out. The stratum corneum provides a barrier to the loss of water from inside the body to the external environment and from attack from the external environment to the interior of the body. Still further, as used herein, “tissue membrane” can refer to an aggregate of cells of a particular kind, together with their intercellular substance, that forms a structural material. Other tissues suitable for use with such devices and compositions include mucosal tissue and soft organs.

As used herein, the term, “subcutaneous fluid” can include, without limitation, moisture, plasma, blood, one or more proteins, interstitial fluid, and any combination thereof. In one aspect, a subcutaneous fluid according to this description is a moisture source comprising water.

As used herein, “transdermal” or “percutaneous” means passage of a permeant into and through the biological membrane to achieve effective therapeutic blood levels or local tissue levels of a permeant, or the passage of a molecule or fluid present in the body (“analyte”) out through the biological membrane so that the analyte molecule maybe collected on the outside of the body.

As used herein, the term “permeant,” “drug,” “permeant composition,” or “pharmacologically active agent” or any other similar term are used interchangeably to refer to any chemical or biological material or compound suitable for transdermal administration by the methods previously known in the art and/or by the methods taught in the present description, that induces a desired biological or pharmacological effect, which may include but is not limited to (1) having a prophylactic effect on the organism and preventing an undesired biological effect such as an infection, (2) alleviating a condition caused by a disease, for example, anti-aging, alleviating pain or inflammation, and/or (3) either alleviating, reducing, or completely eliminating the disease from the organism. The effect may be local, such as providing for a local anesthetic effect, or it may be systemic. Such substances include broad classes of compounds normally delivered into the body, including through body surfaces and membranes, including skin. In general, for example and not meant to be limiting, such substances can include any bioactive agents such as drug, chemical, or biological material that induces a desired biological or pharmacological effect.

As used herein, an “effective” amount of a pharmacologically active agent means a sufficient amount of a compound to provide the desired local or systemic effect and performance at a reasonable benefit/risk ratio attending any medical treatment. An “effective” amount of a permeation or chemical enhancer as used herein means an amount selected so as to provide the desired increase in biological membrane permeability, the desired depth of penetration, rate of administration, and amount of drug delivered.

As used herein, “animal” or “organism” refers to humans and other living organisms including plants, to which embodiments of the present disclosure maybe applied.

As used herein, “analyte” means any chemical or biological material or compound suitable for passage through a biological membrane by the technology taught in this present disclosure, or by technology previously known in the art, of which an individual might want to know the concentration or activity inside the body. Glucose is a specific example of an analyte because it is a sugar suitable for passage through the skin, and individuals, for example those having diabetes, might want to know their blood glucose levels. Other examples of analytes include, but are not limited to, such compounds as sodium, potassium, bilirubin, urea, ammonia, calcium, lead, iron, lithium, salicylates, and the like.

As used herein, “transdermal flux rate” is the rate of passage of any analyte out through the skin of an individual, human or animal, or the rate of passage of any permeant, drug, pharmacologically active agent, dye, or pigment in and through the skin of an organism.

As used herein, “non-invasive” means not requiring the entry of a needle, catheter, or other invasive medical instrument into a part of the body.

As used herein, “minimally invasive” refers to the use of mechanical, hydraulic, or electrical means that invade the stratum corneum to create a small hole or micropore without causing substantial damage to the underlying tissues.

As used herein, “pharmaceutically acceptable carrier” refers to a carrier in which a substance such as a pharmaceutically acceptable drug could be provided for deliver. Pharmaceutically acceptable carriers are described in the art, for example, in “Remington: The Science and Practice of Pharmacy,” Mack Publishing Company, Pennsylvania, 1995, the disclosure of which is incorporated herein by reference. Carriers could include, for example, water and other aqueous solutions, saccharides, polysaccharides, buffers, excipients, and biodegradable polymers such as polyesters, polyanhydrides, polyamino acids, liposomes and mixtures thereof.

As used herein, “reservoir” refers to a designated area or chamber within a device which is designed to contain a permeant for delivery through an artificial opening in a biological membrane into an organism or may be designed to receive a biological fluid sample extracted from an organism through an artificial opening in a biological membrane. A reservoir could also contain excipient compounds which enhance the effect of a separately contained bioactive permeant. Additionally, a reservoir could contain or be treated with reactive enzymes or reagents designed to allow the measurement or detection of a selected analyte in an extracted biological fluid. A reservoir may be comprised of an open volume space, a gel, a flat planar space which has been coated or treated with a selected compound for subsequent release or reaction, or a permeable solid structure such as a pellet, tablet or porous polymer.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’‘preferred,’ ‘desired,’ or ‘desirable,’ and words of similar meaning should not be understood as implying that certain features are critical, essential, or even important to the structure or function of the disclosure, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified. The transitional phrase “consisting of” excludes any element, step, or ingredient not specified in the claim.

### Patch

The devices and methods of the present disclosure can be used to transdermally deliver an active agent across the skin. In some aspects, the patch may comprise a top layer comprising an adhesive, a middle layer comprising an active agent, and a bottom layer. In some embodiments, the bottom layer comprises a release liner. In some embodiments, the middle layer further comprises a carrier. In some embodiments, the transdermal patch may be formulated as a single-layer drug-in-Adhesive patch, multi-layer drug-in-adhesive patch, reservoir matrix, vapour patch, microneedle patch. In some embodiments, the transdermal patch may include one or more type of patches selected from single-layer drug-in-adhesive patch, multi-layer drug-in-adhesive patch, reservoir matrix, vapour patch, and microneedle patch

In some embodiments, the middle layer comprises a matrix. In some embodiments, the matrix is a gel. In some embodiments, the gel is a hydrogel. In some embodiments, the hydrogel is an acrylic hydrogel. Examples of hydrogels include but are not limited to gelled and/or cross-linked water swellable polyolefins, polycarbonates, polyesters, polyamides, polyethers, polyepoxides and polyurethanes such as, for example, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(allyl alcohol). Other suitable polymers include but are not limited to cellulose ethers, methyl cellulose ethers, cellulose and hydroxylated cellulose, methyl cellulose and hydroxylated methyl cellulose, gums such as guar, locust, karaya, xanthan gelatin, and derivatives thereof. For iontopheresis, for example, the polymer or polymers may include an ionizable group such as, for example, (alkyl, aryl or aralkyl)carboxylic, phosphoric, glycolic or sulfonic acids, (alkyl, aryl or aralkyl) quaternary ammonium salts and protonated amines and/or other positively charged species as described in U.S. Pat. No. 5,558,633, which is incorporated herein by reference in its entirety.

In some embodiments, the patch is a pressure-sensitive patch in which the adhesive is the active ingredient carrier. In some embodiments, the pressure-sensitive patch comprises a drug-containing polymer matrix that comprises a pressure-sensitive adhesive or bioadhesive, and is adopted for direct application to a subject's skin. In some embodiments, the pressure-sensitive patch includes a pressure-sensitive adhesive. In other embodiments, transdermal drug delivery system comprises a skin-contacting face adhesive layer separate from the drug-containing polymer matrix. In such embodiments, the polymer matrix optionally may be non-adhesive. In some embodiments, the polymer matrix comprises a pressure-sensitive adhesive polymer or a bioadhesive polymer, with drug dissolved or dispersed therein. In some embodiments the polymer matrix comprises a polymer that may or may not be a pressure-sensitive adhesive polymer, and also comprises tackifiers, plasticizers, crosslinking agents, enhancers, co-solvents, tillers, antioxidants, solubilizers, crystallization inhibitors, or other additives described herein. In some embodiments, the adhesive is an acrylic based adhesive. In some embodiments, the acrylic based adhesive may further include one or more of oleic acid, dipropylene glycol, and sodium polyacrylate. In some embodiments, the acrylic based adhesive further includes a polyester backing. In some embodiments the acrylic based adhesive further includes a polyester release liner.

In some embodiments, the patch includes a pressure-sensitive adhesive or bioadhesive. In some embodiments, the pressure-sensitive adhesive includes the active agent. As used herein, the term “pressure-sensitive adhesive” refers to a viscoelastic material which adheres instantaneously to most substrates with the application of very slight pressure and remains permanently tacky. As noted above, a polymer is a pressure-sensitive adhesive within the meaning of the term as used herein if it has the properties of a pressure-sensitive adhesive per se. Other polymers can be made to function as a pressure-sensitive adhesive by admixture with tackifiers, plasticizers or other additives. The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers, to which they are added. In some embodiments, the adhesive is an acrylic based adhesive. In some embodiments, the acrylic based adhesive may further include one or more of oleic acid, dipropylene glycol, and sodium polyacrylate. In some embodiments, the acrylic based adhesive further includes a polyester backing. In some embodiments the acrylic based adhesive further includes a polyester release liner.

The polymer matrices described herein may be prepared by methods known in the art. For example, a polymer matrix can be prepared by blending the components of the polymer matrix, applying the matrix material to a support layer such as a backing layer or release liner (such as by calender coating, hot melt coating, solution coating, etc.), and removing any remaining solvents. The polymer matrices can be formed into systems by methods known in the art, such as by die-cutting into sizes and shapes suitable for use.

In some embodiments, the patch is a micro-needle patch. In some embodiments, the micro-needle patch includes one or more microneedles. In embodiments, the one or more microneedles have a height from about 100 μm to about 2000 μm, from about 100 μm to about 1500 μm, from about 100 μm to about 1000 μm, or from about 500 μm to about 1000 μm. The one or more microneedles may be arranged on a base substrate in any suitable density. For example, a plurality of microneedles may be arranged in even or staggered rows in an array, wherein each microneedle is separated from its nearest neighboring microneedle by a distance between about 50% and about 200% of the height of the microneedle, (e.g., between about 75% about and about 150% of the height of the microneedle, or by about equal to the height of the microneedle). Any suitable number of microneedles may be used. In one embodiment, a plurality of microneedles may include from 5 to 10,000 microneedles, such as from 50 to 1000 microneedles or from 50 to 200 microneedles.

In some embodiments, the release liner includes a polyester or polyethylene. In some embodiments, the release liner includes a polyester liner. In some embodiments, the release liner includes a polyethylene liner. In some embodiments, the release liner is a paper-based. In some embodiments, the release liner is a polymer coated paper. In some embodiments, the release liner is a polyester film. In some embodiments, the release liner is a silicone based. In some embodiments, the release liner is on either one or both sides of the substrate.

In some embodiments, the adhesive or gel concentration may be from about 0.001 μg/ml to 100 μg/ml. In some embodiments, the adhesive or gel concentration may be from about 0.001 μg/ml, 0.01 μg/ml, 0.1 μg/ml, 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1.0 μg/ml, 3 μg/ml, 5 μg/ml, 7 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, or ranges including and/or spanning the aforementioned values.

In some embodiments, the adhesive or gel has a water holding capacity (WHC) of from about 0.01 mg/cm² to about 100 mg/cm². The water holding capacity of the gel means the amount of moisture the matrix can hold per 1 cm². In some embodiments, the gel water holding capacity is from about 0.5 mg/cm² to about 8 mg/cm². In some embodiments, the gel water holding capacity is from about 1 mg/cm² to about 6 mg/cm². In some embodiments, the gel water holding capacity is from about 2 mg/cm² to about 5 mg/cm². In some embodiments, the gel has a WHC of from about 10% to about 40% by weight. In some embodiments, the gel has a WHC may be 5%, 10%, 15%, 20%, 25%, 30%, 35%, 40%, 45%, 50%, or ranges including and/or spanning the aforementioned values.

The water holding capacity of the adhesive or gel may be controlled by adjusting the thickness and weight of the gel. In some embodiments, the gel thickness is from about 0.01 mm to about 5 mm. In some embodiments, the gel thickness is from about 0.1 mm, 0.2 mm, 0.3 mm, 0.4 mm, 0.5 mm, 0.6 mm, 0.7 mm, 0.8 mm, 0.9 mm. 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, or ranges including and/or spanning the aforementioned values.

In some embodiments, the gel areal weight is about 10 g/m² to about 100 g/m². In some embodiments, the gel areal weight is about 20 g/m² to about 80 g/m². In some embodiments, the gel areal weight is about 30 g/m² to about 70 g/m². In some embodiments, the matrix areal weight is about 40 g/m² to about 60 g/m².

In some embodiments, the gel areal weight is about 0.01 mg/cm² to about 30 mg/cm². In some embodiments, the gel areal weight is about 5 mg/cm² to about 30 mg/cm². In some embodiments, the gel areal weight is about 10 mg/cm² to about 30 mg/cm². In some embodiments, the gel areal weight is about 20 mg/cm² to about 30 mg/cm². In some embodiments, the gel areal weight is about 0.5 mg/cm² to about 5 mg/cm². In some embodiments, the gel areal weight is about 0.5 mg/cm² to about 10 mg/cm².

In some embodiments, the patch covers a skin cumulative coverage area may be from about 1 cm² to about 10,000 cm². In some embodiments, the skin cumulative coverage area may be from about 1 cm², 5 cm², 10 cm², 25 cm², 50 cm², 75 cm², 100 cm², 150 cm², 200 cm², 250 cm², 300 cm², 350 cm², 400 cm², 450 cm², 500 cm², 750 cm², 1000 cm², 2000 cm², 3000 cm², 4000 cm², 5000 cm², 6000 cm², 7000 cm², 8000 cm², 9000 cm², 10,000 cm², or ranges including and/or spanning the aforementioned values.

In some embodiments, the size of the patch is about 0.25 cm² to about 4 cm². In some embodiments, the size of the patch is about 0.5 cm² to about 3 cm². In some embodiments, the size of the patch is about 1 cm² to about 2 cm². In some embodiments, the size of the patch is about 1 cm². In some embodiments, the size of the patch is about 2 cm². In some embodiments, the size of the patch is about 2 cm² by 2 cm². In some embodiments, the size of the patch is about 3 cm² by 3 cm².

In some embodiments, the patch provides a release rate of an active agent from about 0.001 pg (picograms) to 100 ng (nanograms) per hour. In some embodiments, the release rate of the active agent is from about 0.001 pg, 0.002 pg, 0.0044 pg, 0.005 pg, 0.008 pg, 0.010 pg, 0.015 pg, 0.020 pg, 0.025 pg, 0.030 pg, 0.035 pg, 0.040 pg, 0.045 pg, 0.050 pg, 0.075 pg, 0.100 pg, 0.150 pg, 0.200 pg, 0.300 pg, 0.350 pg, 0.400 pg, 0.450 pg, 0.500 pg, 0.550 pg, 0.600 pg, 0.650 pg, 0.700 pg, 0.750 pg, 0.800 pg, 0.850 pg, 0.900 pg, 1.0 pg, 2.0 pg, 3.0 pg, 4.0 pg, 5.0 pg, 6.0 pg, 7.0 pg, 8.0 pg, 9.0 pg, 10 pg, 20 pg, 40 pg, 60 pg, 80 pg, 100 pg, 150 pg, 200 pg, 250 pg, 300 pg, 350 pg, 400 pg, 450 pg, 500 pg, 750 pg, 1000 pg, 1.5 ng, 2 ng, 2.5 ng, 3 ng, 3.5 ng, 4 ng, 5 ng, 7.5 ng, 10 ng, 15 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 150 ng, or ranges including and/or spanning the aforementioned values.

In some embodiments, the top layer includes a non-woven backing. In some embodiments, the non-woven backing includes a non-woven polyester fabric. In some embodiments, the non-woven polyester fabric may be coated with 1.5 ml of acrylic pressure-sensitive adhesive. An example of a non-woven backing is shown in FIG. 3.

### Formulation

In some embodiments, the active agent comprises a histamine receptor modulator. In some embodiments, the histamine receptor modulator activates histamine H1 receptors. In some embodiments, the histamine receptor modulator activates histamine H2 receptors. In some embodiments, the histamine receptor modulator activates histamine H3 receptors. In some embodiments, the histamine receptor modulator activates histamine H4 receptors. In some embodiments, the histamine receptor modulator activates the histamine H1, H2, H3 and H4 receptors at various rates/levels over the application/exposure time. In some embodiments, the histamine receptor modulator is histamine. In some embodiments, the histamine receptor modulator is a histamine salt. In some embodiments, the histamine receptor modulator is N-alpha methyl histamine. In some embodiments, the histamine salt is selected from the group comprising of histamine diphosphate, histamine phosphate, and histamine dihydrochloride. In some embodiments, the active agent comprises other histamine agonists. Combinations of these salts or other various salts of histamine may also be used, depending on the embodiment. In some embodiments, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof.

In some embodiments, the carrier may be selected from comprises water, saline, saccharides, polysaccharides, buffers, excipients, biodegradable polymers, liposomes, stabilizer, skin irritation reducer, and mixtures thereof. In some embodiments, the pharmaceutically acceptable carrier includes water and other aqueous solutions, saccharides, polysaccharides, buffers, excipients, and biodegradable polymers such as polyesters, polyanhydrides, polyamino acids, liposomes and mixtures thereof.

In some embodiments, the carrier comprises a silicone-based polymer. The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives. Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989). Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series, such as -4502, and -4600 series.

In some embodiments, the middle layer further includes a pharmaceutically acceptable liquid carrier. The liquid carrier or vehicle may be a solvent or liquid dispersion medium comprising, for example, water, saline solution, ethanol, a polyol, vegetable oils, nontoxic glyceryl esters, and suitable mixtures thereof. The solubility of a chemical blocking agent may be enhanced using solubility enhancers such as, for example, water; diols, such as propylene glycol and glycerol; mono-alcohols, such as ethanol, propanol, and higher alcohols; DMSO (dimethylsulfoxide); dimethylformamide, N,N-dimethylacetamide; 2-pyrrolidone, N-(2-hydroxyethyl)pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones). The proper fluidity may be maintained, for example, by the formation of liposomes, by the maintenance of the required particle size in the case of dispersions or by the use of surfactants.

In some embodiments, the middle layer further comprises a permeation enhancing agent. In some embodiments, the permeation-enhancing agent may be selected from the group comprising dimethylsulfoxide, combinations of sucrose fatty acid esters with a sulfoxide or phosphoric oxi, or eugenol.

In some embodiments, the middle layer further comprises an absorption enhancing agent. In some embodiments, the absorption enhancing agent is selected from the group consisting of sulphoxides, alcohols, polyols, alkanes, fatty acids, esters, amines, amides, terpenes, surfactants, cyclodextrins, dimethylsulphoxide, pyrrolidones, N,N-diethyl-m-toluamide, and laurocapram.

In some embodiments, the skin irritation reducer is an organic acid. In some embodiments, the skin irritation reducer is a salt form of the organic acid. In some embodiments, the organic acid is selected from ascorbic acid, citric acid, succinic acid, tartaric acid, maleic acid, lactic acid, benzoic acid, and sorbic acid or a combination thereof. In some embodiments, the skin irritation reducer is selected a non-organic acid. In some embodiments, the skin irritation reducer is a salt form of the non-organic acid. In some embodiments, the non-organic acid is hydrochloric acid, phosphoric acid, boric acid, acetic acid or a combination thereof. In some embodiments, the non-organic acid is evaporated during the manufacturing process.

In some aspects, the stabilizer is a saccharide. In some embodiments, the saccharide is selected from mannitol, maltose, trehalose, xylitol, xylose, dextrose, lactose, sorbitol, sucrose, fructose, maltitol, erythritol, lactitol, isomalt, and cyclodextrin or a combination thereof. In some embodiments, the stabilizer is sucrose.

In some embodiments, the middle layer comprises about 0.1% to about 90% by weight of an active agent. In some embodiments, the active agent comprises approximately 20 weight % to approximately 80 weight % of the middle layer, including amounts such as 25 weight %, 30 weight %, 35 weight %, 40 weight %, 45 weight %, 50 weight %, 55 weight %, 60 weight %, 65 weight %, 70 weight %, and 75 weight % of the middle layer, or ranges including and/or spanning the aforementioned values.

In some embodiments, the middle layer comprises an active agent from about 10 ng to 700 μg. In some embodiments, the middle layer comprises about 10 ng, 100 ng, 1 μg, 10 μg, 20 μg, 30 μg, 40 μg, 50 μg, 100 μg, 150 μg, 200 μg, 250 μg, 300 μg, 400 μg, 500 pg, 600 μg, 700 μg, 800 μg, 900 μg, 1000 μg, or ranges including and/or spanning the aforementioned values.

In some aspects, the middle layer comprises from about 0.001 μg/ml to about 100 μg/ml active agent. In some embodiments, the middle layer comprises from about 0.001 μg/ml to about 3.0 μg/ml active agent. In some embodiments, the middle layer comprises from about 20 μg/ml to about 500 μg/ml active agent. In some embodiments, the middle layer comprises from about 50 μg/ml to about 300 μg/ml active agent. In some embodiments, the middle layer comprises from about 2 μg/ml to about 50 μg/ml active agent. In some embodiments, the middle layer comprises about 2.7 μg/ml active agent. In some embodiments, the middle layer comprises about 50 μg/ml active agent. In some embodiments, the middle layer comprises about 20 μg/ml active agent.

In some aspects, the middle layer of the patch comprises a carrier. In some embodiments, the carrier comprises about 0.1% to about 90% by weight of the middle layer. In some embodiments, the active agent comprises approximately 20 weight % to approximately 80 weight % of the middle layer, including amounts such as 25 weight %, 30 weight %, 35 weight %, 40 weight %, 45 weight %, 50 weight %, 55 weight %, 60 weight %, 65 weight %, 70 weight %, and 75 weight % of the middle layer, or ranges including and/or spanning the aforementioned values.

In some embodiments, the middle layer comprises about 0.1 to about 90% by weight of a stabilizer. In some embodiments, the stabilizer comprises approximately 20 weight % to approximately 80 weight % of the middle layer, including additional amounts as 25 weight %, 30 weight %, 35 weight %, 40 weight %, 45 weight %, 50 weight %, 55 weight %, 60 weight %, 65 weight %, 70 weight %, and 75 weight % of the middle layer, or ranges including and/or spanning the aforementioned values.

In some embodiments, the pH of the middle layer is from about 1.5 to about 8. In some embodiments, the pH of the middle layer is from about 4 to about 6. In some embodiments, the pH of the middle layer is from about 5 to about 6. In some embodiments, the pH of the middle layer is about 3. In some embodiments, the pH of the middle layer is about 4. In some embodiments, the pH of the middle layer is about 5. In some embodiments, the pH of the middle layer is about 6.

In some aspects, the patch further comprises an anti-microbial agent. In some embodiments, the anti-microbial agent is selected from benzoic acid, methyl paraben, propyl paraben, benzalkonium chloride, chlorhexidine, cresol, salicylic acid, sorbic acid, benzetonium chloride, chlorobutanol, phenol, thimerosal, and combinations thereof.

In some aspects, formulation as described herein is formulated for topical application. In some embodiments, the topical application is in the form of a cream. In some embodiments, the formulation is in the form of a gel. In some embodiments, the formulation is in the form of a lotion. In some embodiments, the formulation is the form of a dispersed liquid, such as a spray or mist. Topical formulations may generally be comprised of a pharmaceutical carrier, co-solvent, emulsifier, penetration enhancer, preservative system, and emollient. In some embodiments, the formulation as described herein is formulated as a pill or a tablet. In some embodiments, the pill or tablet may have a similar time released profile as a patch described herein.

In some embodiments, the formulation is in the form of a propellant, such as a spray.

A “penetration enhancer” such as glycol or glycerin are such agents known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer. The penetration enhancer can be used to obtain the desired flux profile over time.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the transdermal patch contains a dosage of active ingredient anywhere from 1 ng to about 700 μg. In some embodiments, the transdermal patch contains about ing, 10 ng, 100 ng, 1 μg, 10 μg, 20 μg, 30 μg, 40 μg, 50 μg, 100 μg, 150 μg, 200 μg, 250 μg, 300 μg, 400 μg, 500 μg, 600 μg, 700 μg, 800 μg, 900 μg, 1000 μg, or ranges including and/or spanning the aforementioned values.

### Systems

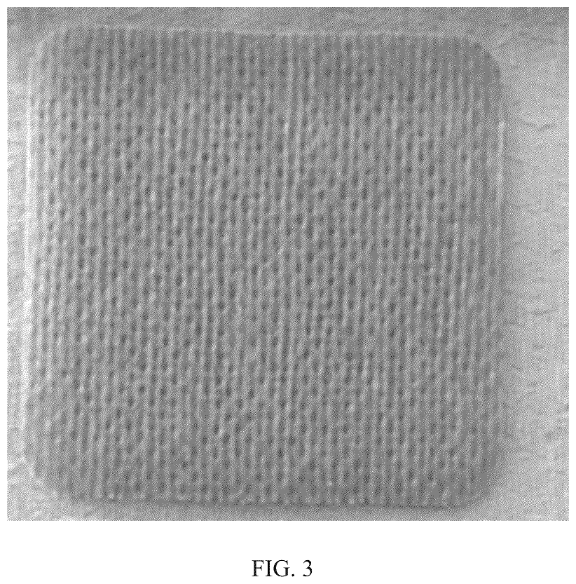

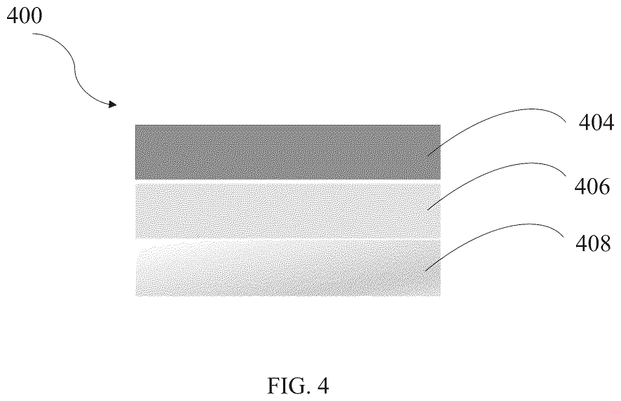

In aspects, a transdermal delivery system for delivering at least one drug through the skin are provided. In some embodiments, the system includes a patch. In some embodiments, the system includes an applicator configured to supply a drug delivery patch and a patch. In some embodiments, the patch includes a top layer comprising an adhesive, a middle layer with at least one drug disposed within the middle layer, and a bottom layer, wherein the bottom layer comprises a release liner. In some embodiments, disposed within the middle layer is at least one drug that dissolves in an exudate, for example, an excipient, stabilizer, pH adjuster, buffer, preservative, antiseptic, solubility enhancer, thickener, antioxidant, transdermal absorption enhancer, irritation modifier, chelating agent, or the like. In some embodiments, the middle layer contains a sufficient amount of a histamine modulator sufficient to induce or maintain a desired histamine level in a subject. Accordingly, referring to FIG. 4, a transdermal delivery system is generally indicated at 400. The patch includes a top layer 402, a middle layer 404, and a bottom layer 406. In some embodiments, the middle layer 404 may be a liquid or gel. In some embodiments, the middle layer 404 may include a reservoir. In some embodiments, the middle layer 404 may include a matrix carrier that can be self-adhesive or non-adhesive. In some embodiments, the top layer 402 includes an adhesive. In some embodiments, the bottom layer 406 includes a release liner.

In some embodiments, the top layer 402 includes a drug-permeable adhesive that is applied over the middle layer 404. In some embodiments, a further layer can be employed as a rate controlling membrane, where the layer is selected to provide for selective movement of the histamine modulator through the layer. The rate controlling membrane can be used to provide control over delivery of a histamine modulator from the system. Despite such a high degree of system control in the present systems, certain systems are able to provide a net flux of the histamine modulator from the system through the skin of at least about 0.001 ng/cm²/hour to at least about 500 μg/cm²/hour.

In some embodiments, bottom layer 406 can adhere to the drug-containing middle layer 404 and function as the primary structural element of the patch. The bottom layer 406 is thus typically a sheet or film of a preferably flexible elastomeric material that is substantially impermeable to the histamine modulator. In some embodiments, the bottom layer 406 may have a thickness from about 0.1 mm to about 5.0 mm. In some embodiments, the bottom layer 406 material may permit the patch to follow the contours of the skin such that it can be worn comfortably on any skin area including joints or other areas of flexure. Accordingly, there is a reduced likelihood of the patch dislodging from the skin due to differences in the flexibility or resiliency of the skin and the device, as well as in response to normal mechanical strain brought about by movement and the like. The bottom layer 406 may further be a monolithic (single layer) or a multi-layer (multi-laminate), and may further be a breathable or occlusive material comprising fabric. In some embodiments, the bottom layer 406 will be a polymeric material, or a laminate of polymeric materials. Suitable materials include, but are not limited to, polyethylene, polypropylene, polyesters, polyurethanes, polyethylene vinyl acetate, polyvinylidene chloride, block copolymers such as PEBAX, polyvinyl acetate, polyvinylidene chloride, polyurethane, ethylene vinyl acetate, polyethylene terephthalate, polybutylene terephthalate, coated paper products, metal or metalized sheets and the like, and any combinations thereof.

In some embodiments, the bottom layer 406 comprises a low-, medium- or high-density polyethylene material, or a polyester material. In some embodiments, the bottom layer 406 includes a laminate of polyethylene and aluminum vapor coated polyester (e.g., SCOTCHPAK® 1109 Backing, available from 3M, St. Paul, Minn.), or a laminate of polyester and polyethylene/ethylene vinyl acetate (e.g., SCOTCHPAK® 9733 Backing, available from 3M).

In some embodiments, the middle layer 404 is disposed on the bottom layer 406. The middle layer may be formed from any number of standard materials well-known in the art. In those devices where the middle layer 404 is a liquid or gel-type reservoir, any suitable gelling agent may be used to form an aqueous gel system, for example a hydrogel. In some embodiments, the reservoir is a matrix-type reservoir. In some embodiments, the matrix-type reservoir may be formed from any polymeric material in which a histamine modulator has some solubility within a desired solubility range, for example, a polyurethane, ethylene/vinyl acetate copolymer (EVA), polyacrylate, styrenic block copolymer, and the like.

In some embodiments, the transdermal delivery system may be a monolithic, adhesive matrix device and the middle layer 404 can be formed from standard pressure sensitive adhesives known in the art. Suitable pressure sensitive adhesives for use in the practice of the embodiments in this disclosure thus include, but are not limited to, polyacrylates, polysiloxanes, polyisobutylene (PIB), polyisoprene, polybutadiene, styrenic block polymers, blends and combinations of the above, and the like. Suitable styrenic block copolymer-based adhesives include, but are not limited to, styrene-isoprene-styrene block copolymer (SIS), styrene-butadiene-styrene copolymer (SBS), styrene-ethylenebutene-styrene copolymers (SEGS), and di-block analogs thereof. Suitable acrylic polymers are comprised of a copolymer or terpolymer comprising at least two or more exemplary components selected from acrylic acids, alkyl acrylates, methacrylates, copolymerizable secondary monomers or monomers with functional groups. Examples of monomers include, but are not limited to, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, and the like. See, e.g., Satas (1989) “Acrylic Adhesives,” Handbook of Pressure-Sensitive Adhesive Technology, 2nd ed., pp. 396-456 (D. Satas, ed.), Van Nostrand Reinhold, N.Y. In a preferred embodiment, the pressure-sensitive adhesive is an acrylate having no functional groups or cross linkers (e.g., DURO-TAK® 87-9301, available from National Starch & Chemical, Bridgewater, N.J.), or a blend of acrylate-vinylacetates having —COH and OH functional groups (DURO-TAK® 87-2051 and 87-2287, National Starch & Chemical).

In some embodiments, the middle layer 404 may be provided as either a liquid or gel reservoir-type or a matrix-type device. Both of these configurations will naturally include a bottom layer 406 that provides a protective outer surface for the devices, as well as a release liner or layer that will cover the adhesive portion of the device that is used to affix the same to the skin of a subject. The release liner may be removed prior to application, thereby exposing the adhesive portion of the device. In some embodiments, the adhesive portion may be a pressure-sensitive adhesive.

In some embodiments, the middle layer 404 includes a histamine modulator. In some embodiments, the middle layer 404 may further include other optional ingredients, such as carriers, vehicles, additives, excipients, stabilizers, dye, diluents, plasticizers, tackifying agents, crystallization inhibitors, solubility enhancers, inert fillers, antioxidants, anti-irritants, vasoconstrictors and other materials without pharmacological activity that are suitable for administration in conjunction with the transdermal delivery system. In some embodiments, the additional materials may include a pharmaceutically acceptable materials that are nontoxic, do not interfere with delivery of a histamine modulator from the system, and are not for any other reasons biologically or otherwise undesirable. If a pressure sensitive adhesive is used in accordance with the present disclosure, this must also be pharmaceutically acceptable. Examples of illustrative materials include water, mineral oil, silicone, inorganic gels, aqueous emulsions, liquid sugars, waxes, petroleum jelly, and a variety of other oils and polymeric materials.

In some embodiments, the middle layer 404 includes an adhesive matrix. In some embodiments, the middle layer 404 includes one or more materials capable of improving its adhesive characteristics such as by reducing quick tack (tackifying agents), reducing cold-flow, increasing viscosity, and/or toughening the matrix structure. Examples of suitable materials include, but are not limited to, aliphatic hydrocarbons; aromatic hydrocarbons; hydrogenated esters; polyterpenes; polybutenes, silicone dioxide, silica, hydrogenated wood resins; tackifying resins, aliphatic hydrocarbon resins made from cationic polymerization of petrochemical feedstocks or the thermal polymerization and subsequent hydrogenation of petrochemical feedstocks, rosin ester tackifiers, mineral oil, polybutylmethacrylate, high molecular weight acrylates, and any combinations thereof.

In some embodiments, the patch further includes a reservoir. In some embodiments the reservoir contains a sufficient amount of a histamine modulator to induce or maintain a desired histamine level in a subject. In some embodiments, the reservoir may be an adhesive or non-adhesive matrix. In some embodiments, the reservoir may be a non-adhesive matrix. In some embodiments, the reservoir includes a dry non-hydrated thickness of about 1 mm to about 5 mm.

In some embodiments, a middle layer 404 includes a viscosity-enhancing agent is added to a pressure-sensitive material such as a PIB or acrylic adhesive base. Due to the low hydrophobicity and low surface tension of the SAIB material, this enables the resultant adhesive/viscosity agent mixture to retain pressure sensitive properties even after the system has been applied and removed from the skin surface a number of times. This in turn allows the subject wearing a long-duration patch to remove the device during showering or heavy exercise, and then reapply the device without losing adhesion.

In some embodiments, the middle layer 404 can further comprise a plasticizer material that is typically an inert, organic, apolar, nonvolatile hydrophobic liquid. In some embodiments, the plasticizer may be a hydrophobic liquid. Suitable plasticizer materials thus include, but are not limited to, various long-chain aliphatic esters and alcohols, including such materials as polybutene, mineral oil, linseed oil, octyl palmitate, squalene, squalane, silicone oil, isobutyl stearate, olive oil, isopropyl myristate, isostearyl alcohol, oleyl alcohol, and the like. Particularly preferred for use herein is polybutene, for example IDOPOL® L-14 or H-100, available from BP Amoco, Naperville, Ill.), having a viscosity substantially equivalent to light mineral oil.

In some embodiments, the middle layer 404 can include one or more filler materials. Suitable fillers include, but are not limited to, metal oxides, inorganic salts, synthetic polymers, clays and the like. The metal oxides may be silicon dioxide, zinc oxide, magnesium oxide, titanium oxide, and calcium oxide. Inorganic salts can be calcium, magnesium and sodium carbonate, calcium and magnesium sulfate, calcium phosphate, and the like. Synthetic polymers can include methacrylic resin, nylon, polyethylene, and the like. Suitable clay compounds include talc, bentonite and kaolin.

In some embodiments, the release liner may be formed from a material impermeable to a histamine modulator and other components of the system, and is easily removable from the middle layer 404. Release liners can generally be made of the same materials as the backing layer. Suitable materials thus include a polymeric material that may be optionally metallized. Examples of the polymeric materials include polyurethane, polyvinyl acetate, polyvinylidene chloride, polypropylene, polycarbonate, polystyrene, polyethylene, polyethylene terephthalate, polybutylene terephthalate, paper, and the like, and a combination thereof. In preferred embodiments, the protective layer comprises a siliconized polyester sheet, or has a fiuoropolymer coating. Example materials include, but are not limited to, SCOTCHPAK® 9744 (available from 3M), and MEDIRELEASE® 2249 (available from Mylan Tech., St. Paul, Minn.).

In some embodiments, the transdermal drug delivery system described herein may provide for improved patient compliance and enhanced drug delivery capabilities. Embodiments of the transdermal drug delivery system may also provide for reduced risks of adverse effects caused by uncontrolled delivery and reduce the development terms and costs for drugs for patients. Embodiments of the transdermal drug delivery system as described herein may be used for patients with a wide range of skin types, conditions, and so forth with a lower variation on individual drug delivery results. Embodiments of the transdermal drug delivery system may work with a wide variety of drug formulations, for example high potency drugs, high molecular weight drugs, and biologics, among others. Embodiments of the transdermal drug delivery system also enables delivery of the drugs with higher utilization as compared to current transdermal patches in an efficient and controlled manner, improving bioavailability of the drug in the patient's body.

In some embodiments, the transdermal delivery system may be configured as a monolithic device where a histamine modulator is contained in the middle layer 404 adhered to a top layer 406. In some embodiments, the monolithic transdermal delivery system for delivering a histamine modulator through the skin is provided.

In some embodiments, the applicator is configured to supply a drug delivery patch. In some embodiments, the applicator is a microneedle drug delivery applicator. In some embodiments, the applicator is a disposable cartridge having micro skin penetrating members serving as microneedles configured to pierce the patch and the skin of a subject. In some embodiments, the microneedles are configured as a microneedle array.

In some embodiments, the transdermal drug delivery system further comprises a control unit. The control unit may be a hardware device or circuit that can implement the various components described herein and provide the control and power to one or more applicators of the transdermal drug delivery system. In some embodiments, the control unit includes a processor which controls operation of the control unit. In some embodiments, the processor may also be referred to as a central processing unit (CPU) or hardware processor. In some embodiments, the control unit may also include a memory unit, which may provide instructions and/or data to the processor and may serve as a repository for storage of instructions and/or data from the processor. In some embodiments, the processor may comprise or be a component of a processing system implemented with one or more processors. In some embodiments, the control unit may include networking components, for example, a transmitter and a receiver to allow transmission and reception of data between the control unit and a remote location or device.

In some embodiments, the applicator is configured to supply a drug delivery patch. In some embodiments, the applicator is a microporation drug delivery applicator. Microporation (for example using laser, RF, and/or heating elements) may be controlled based on changing numbers of pores, pulse duration (quality of pores), and treatment area. However, variations in skin (for example, between different patients, between different locations on a patient's body, an environment of the patient, characteristics of the patient's skin, and so forth) may result in differences in micropore creation (regardless of the microporation method implemented) in different conditions or patients. Such differences may result in ineffective, inconsistent, unsafe, and/or variable drug delivery using transdermal patches in conjunction with known methods of microporation. In some embodiments, the microporation utilizes thermal ablation to create the micropores. Though the examples provided in this disclosure may focus on microporation via such thermal ablation, the systems and methods described herein apply equally to other microporation methods and systems (for example, RF, laser, and so forth).

### Methods

In some aspects, the present disclosure also includes methods for using a patch or system as described herein to administer an active agent to a subject in need thereof. In some embodiments, a method of treating a subject comprises: identifying a subject and applying the patch or system described herein to the subject's skin for a period of time. In some embodiments, the patch comprises a top layer comprising an adhesive, a middle layer comprising an active agent, and a bottom layer.

In some embodiments, the method includes selective activation of a histamine receptor. In some embodiments, the histamine receptor is selected from H1, H2, H3, and H4. In some embodiments, the active agent includes histamine, a histamine salt, or other therapeutic agents that bind or block these receptors, or increase or decrease the ability of histamine (endogenous or exogenous histamine) to bind to the receptors are used in several embodiments. In some embodiments, the method includes the downregulation of the H1 receptor and the upregulation of one, two or all of the H2, H3 and H4 receptor. In some embodiments, the method includes an active agent that regulates the endogenous histamine system. These therapeutic agents may be provided in dosing schedules as described herein. Thus, instead of (or in addition to) affecting histamine receptors directly, an active agent may increase or decrease the manufacture, release, modification, uptake or degradation of histamine is provided. In one embodiment, selective histamine reuptake inhibitors are used. In some embodiments, therapeutic agents that up-regulate or down-regulate methyltransferase and/or diamine oxidase are used to decrease or increase endogenous histamine.

In some embodiments, the transdermal patch includes a secondary active agent. In several embodiments, a secondary active agent includes a compound capable of regulating endogenous GABA are provided, which in turn affects endogenous release of histamine. In one embodiment, histamine release is modulated by endogenous GABA through GABA-A and/or GABA-B receptors. In one embodiment, therapeutic agents that regulate GABA-B receptors or other receptors located on the histaminergic nerve terminals that modulate histamine release (e.g., pre-synaptically or post-synaptically) are provided. In some embodiments, a method is provided for restoring histamine balance in a subject by normalizing activity of histamine receptors through multiple doses administered in successive therapy segments, comprising administering a first therapy segment, that first therapy segment comprising activating a histamine H3 receptor by administering to a subject a first dose of a histamine receptor activator. In some embodiments, the method includes activating a histamine H4 receptor by administering to a subject a second dose of the histamine receptor activator. In some embodiments, the method includes activating a histamine H1 receptor by administering to a subject a third dose of the histamine receptor activator. In some embodiments, the method includes activating a histamine H2 receptor by administering to a subject a fourth dose of the histamine receptor activator. In some embodiments, the method includes administering a second therapy segment, comprising administering the histamine receptor activator in an amount greater than the amount administered in the first therapy segment. In some embodiments, the method includes administering a third therapy segment comprising administering the histamine receptor activator in an amount greater than the amount administered in the second therapy segment.

In additional embodiments, there is also provided a method for restoring histamine balance in a subject by normalizing activity of histamine receptors through multiple doses administered in successive therapy segments comprising administering a first therapy segment, wherein the first therapy segment comprising activating one or more of a histamine H3 receptor and a histamine H4 receptor by administering to a subject a first dose of a histamine receptor activator. In some embodiments, the method includes activating a one or more of a histamine H3 receptor and a histamine H4 receptor by administering to a subject a second dose of the histamine receptor activator. In some embodiments, the method includes activating a histamine H1 receptor by administering to a subject a third dose of the histamine receptor activator, activating a histamine H2 receptor by administering to a subject a fourth dose of the histamine receptor activator. In some embodiments, the method includes administering a second therapy segment, comprising administering the histamine receptor activator in an amount greater than the amount administered in the first therapy segment, and administering a third therapy segment, comprising administering the histamine receptor activator in an amount greater than the amount administered in the second therapy segment. In several embodiments, the increasing amount of the histamine receptor activator administered in each successive therapy segment suppresses activity of the histamine H1 receptor and enhances activity of a histamine receptor selected from the group consisting of the histamine H2, H3, and H4 receptors, thereby normalizing the activity of the histamine receptors and restoring histamine balance.

In several embodiments, there is also provided a method for restoring histamine balance in a subject by normalizing activity of histamine receptors through multiple doses administered in successive therapy segments, comprising administering a first therapy segment to the subject, the first segment comprising administering to a subject a first dose of a therapeutic agent that binds a histamine H3 receptor by administering to the subject a first dose of a therapeutic agent. In some embodiments, the method includes administering to the subject a second dose of a therapeutic agent that binds a histamine H4 receptor by administering to the subject a second dose of the therapeutic agent. In some embodiments, the method includes administering to the subject a first dose of a therapeutic agent that binds a histamine H1 receptor by administering to the subject a third dose of the therapeutic agent, administering to the subject a first dose of a therapeutic agent that binds a histamine H2 receptor by administering to the subject a fourth dose of the therapeutic agent. In some embodiments, the method includes administering a second therapy segment to the subject, the second segment comprising administering the therapeutic agent in an amount greater than the amount administered in the first therapy segment. In some embodiments, the method includes administering a third therapy segment to the subject, the third segment comprising administering the therapeutic agent in an amount greater than the amount administered in the second therapy segment.

In some embodiments, a method for restoring histamine balance in a subject by normalizing activity of histamine receptors through multiple doses administered in successive therapy segments is provided. In some embodiments, the method includes administering a first therapy segment to the subject, the first segment comprising administering to a subject a first dose of a therapeutic agent that binds a histamine receptor selected from the group consisting of a histamine H3 receptor and a histamine H4 receptor by administering to the subject a first dose of a therapeutic agent. In some embodiments, the method includes administering to the subject a second dose of an active agent that binds a histamine receptor selected from the group consisting of a histamine H3 receptor and a histamine H4 receptor by administering to the subject a second dose of the therapeutic agent. In some embodiments, the method includes administering to the subject a first dose of a therapeutic agent that binds a histamine H1 receptor by administering to the subject a third dose of the therapeutic agent. In some embodiments, the method includes administering to the subject a first dose of a therapeutic agent that binds a histamine H2 receptor by administering to the subject a fourth dose of the therapeutic agent. In some embodiments, the method includes administering a second therapy segment to the subject, the second segment comprising administering the therapeutic agent in an amount greater than the amount administered in the first therapy segment. In some embodiments, the method includes administering a third therapy segment to the subject, the third segment comprising administering the therapeutic agent in an amount greater than the amount administered in the second therapy segment.

In some embodiments, a method for down-regulating a histamine H1 receptor is provided. In some embodiments, the method includes administering a first therapy segment, comprising administering to a subject a first dose of an active agent that binds a histamine H3 receptor administering to the subject a first dose of the therapeutic agent. In some embodiments, the method includes administering to the subject a second dose of a therapeutic agent that binds a histamine H4 receptor by administering to the subject a second dose of the therapeutic agent. In some embodiments the method includes administering to the subject a first dose of a therapeutic agent that binds a histamine H1 receptor by administering to the subject a third dose of the therapeutic agent. In some embodiments, the method includes administering to the subject a first dose of a therapeutic agent that binds a histamine H2 receptor by administering to the subject a fourth dose of the therapeutic agent. In some embodiments, the method includes administering to the subject a second therapy segment, the second segment comprising administering the therapeutic agent in an amount greater than the amount administered in the first therapy segment. In some embodiments, the method includes administering a third therapy segment to the subject, the third segment comprising: administering the active agent in an amount greater than the amount administered in the second therapy segment, wherein the increasing amount of the therapeutic agent administered in each successive therapy segment down-regulates activity of the histamine H1 receptor. Optionally, additional therapy segments can be administered as well.

In several embodiments, the histamine receptor activator is administered in one or more of varied concentration (e.g., increased concentration as compared to a preceding administration), varied volume (e.g., increased concentration as compared to a preceding administration), or with a varied timing (e.g., the frequency of administration is reduced with successive segments). The tri-variable approach is beneficial in several embodiments, because cellular distribution and receptor contact can be adjusted. In this manner, selective activation of receptors can be achieved. Moreover, in several embodiments, the sequence of receptor activation can be altered depending on the condition being treated. For example, in several embodiments, a therapeutic agent can be administered at a dose sufficient to preferentially activate or bind an H1 receptor, and increase histamine levels in a subject (e.g., to treat histapenia). Alternatively, a therapeutic agent can be administered at a dose sufficient to preferentially activate or bind H2 and/or H3 receptor (which in several embodiments suppresses the activity and/or expression of the histamine H1 receptor and leads to reduction in histamine levels, such as when treating histadelia).

In some embodiments, each of the therapy segments have an individual dose that is administered to the subject. In some embodiments, each of the therapy segments has a different dose based on the need of the subject. For example, a subject is administered a first therapy segment with 30 μg/ml of histamine hydrochloride and a second therapy segment with 2.7 μg/ml. In some embodiments, the method includes a regimen that comprises a series of transdermal patches ranging from about 20 ng to about 500 μg of histamine hydrochloride for a therapy segment and additional series of transdermal patches of 100 ng to about 300 μg of histamine hydrochloride. It is contemplated that the amounts can vary as described herein.

In some embodiments, the active agent is a histamine or a histamine salt. In some embodiments, the amount of histamine or a histamine salt administered in the body through the skin is about 1 ng to about 10,000 ng. In some embodiments, the amount of histamine or a histamine salt administered in the body through the skin is about 10 ng to about 1000 ng. of histamine or a histamine salt administered in the body through the skin is about 100 ng to about 1000 ng. In some embodiments, the amount administered depends on indication and patient tolerance.

In some embodiments, an active agent may be provided in a transdermal patch. In several embodiments, the first dose of the active agent in the first therapy segment has a concentration ranging from between about 0.001 μg/mL to about 100 μg/mL. In some embodiments, the amount of active agent includes from about 0.5 μg/mL, about 1 μg/mL, about 2 μg/mL, about μg/mL, about μg/mL, about 5 μg/mL, about 6 μg/mL, about 7 μg/mL, about 8 μg/mL, about 9 μg/mL, about 10 μg/mL, or ranges including and/or spanning the aforementioned values.

In several embodiments, the second dose of the histamine receptor activator in the first therapy segment has a concentration ranging from between about 0.1 μg/mL to about 10 μg/mL, including about 0.5 μg/mL, about 1.0 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, or ranges including and/or spanning the aforementioned values.

In several embodiments, the third dose of the histamine receptor activator in the first therapy segment has a concentration ranging from between about 0.001 μg/mL to about 30 μg/mL. In some embodiments, the third dose includes doses from about 0.001 μg/mL, about 0.01 μg/mL, about 0.5 μg/mL, about 1 μg/mL, about 1.5 μg/mL, about 20 μg/mL, about 25 μg/mL, about 30 μg/mL, or ranges including and/or spanning the aforementioned values.

In several embodiments, the fourth dose of the histamine receptor activator in the first therapy segment has a concentration ranging from between about 0.001 μg/mL to about 20 μg/mL, including about 3 μg/mL, about 3.5 μg/mL, about 4 μg/mL, about 4.5 μg/mL, about 5 μg/mL, about 6 μg/mL, about 7 μg/mL, about 8 μg/mL, about 9 μg/mL, about 10 μg/mL, and concentrations therebetween. In several embodiments, the fourth dose of the histamine receptor activator in the first therapy segment has a concentration ranging from between about 0.35 μg/mL to about 10 μg/mL, including about 0.35 μg/mL, about 0.75 μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 6 μg/mL, about 7 μg/mL, about 8 μg/mL, about 9 μg/mL, about 10 μg/mL, or ranges including and/or spanning the aforementioned values.

In some embodiments, the method may be used to treat imbalances of histamine. In some embodiments, the imbalance of histamine may be determined by sub-optimal or supra-optimal histamine levels in the bloodstream and/or urine, and/or other symptoms or characteristics of histamine imbalance. In some embodiments, the method includes a method of lowering histamine levels in a subject having a histamine level above a level required for optimum histamine function comprising administering an active agent as described herein to a subject according to a dosing regimen as disclosed herein.

Additionally, in several embodiments, methods for reducing, ameliorating, preventing, and/or inhibiting the frequency, duration and/or intensity of migraine headaches in a subject through normalization of the activity and/or expression of one or more histamine receptors are provided. In some embodiments, the subject is administered a transdermal patch as described herein. In some embodiments, the method includes identifying a subject susceptible to migraine headaches. In some embodiments, the method includes identifying a subject having a histamine imbalance. In some embodiments, the method includes administering to the subject a first dosing segment comprising a first, a second, a third, and/or a fourth dose of an active agent with a transdermal patch as described herein.

In several embodiments, a method for restoring histamine balance is provided. In some embodiments, normalizing activity of histamine receptors is achieved through administration of one or more transdermal patches as described herein. In several embodiments, the methods comprise administering at least a first, a second, and a third initial therapy segments, each of the initial segments comprising activating a histamine H1, H2, H3 or H4 receptor by administering to a subject a first dose of an active agent as described herein. In several additional embodiments, a method for restoring histamine balance is provided, the method comprising normalizing activity of histamine receptors through multiple doses administered in successive therapy segments by administering successive transdermal patches.

In some embodiments, method for reducing, ameliorating, preventing and/or inhibiting the frequency, duration and/or intensity of Parkinson's Disease, Alzheimer's Disease, coronary disease, leukemia, amyotrophic lateral sclerosis, epilepsy, histadelia, histapenia, and/or one or more conditions associated with aging, and other disorders are provided. In several embodiments, there is additionally provided a transdermal patch as described herein for use in the treatment or prevention of migraine headaches, Parkinson's Disease, Alzheimer's Disease, coronary disease, leukemia, amyotrophic lateral sclerosis and/or epilepsy, histadelia, histapenia, and/or one or more conditions associated with aging, by administration of a dosage of an active agent as described herein. Also provided herein is a transdermal patch as described herein for use in the treatment or prevention of migraine headaches, Parkinson's Disease, Alzheimer's Disease, coronary disease, leukemia, amyotrophic lateral sclerosis, epilepsy, histadelia and/or histapenia (or other ailments) in patients showing an elevated amount of circulating histamine as well as ailments in which patients show a reduced amount of circulating histamine. Thus, in several embodiments, the disclosure comprises a transdermal patch for regulating endogenous histamine levels, including but not limited to use in the treatment or prevention of migraine headaches, Parkinson's Disease, Alzheimer's Disease, coronary disease, leukemia, amyotrophic lateral sclerosis, epilepsy, histadelia, histapenia, and/or one or more conditions associated with aging to restore histamine balance in the subject.

In several embodiments, the disclosure comprises a transdermal patch for use in the treatment or prevention of migraine headaches, Parkinson's Disease, Alzheimer's Disease, coronary disease, leukemia, amyotrophic lateral sclerosis, epilepsy, histadelia, histapenia, and/or one or more conditions associated with aging, by activating or inhibiting the activity and/or expression of a histamine receptor selected from the group consisting of the histamine H1 receptor, the histamine H2 receptor, the histamine H3 receptor, and the histamine H4 receptor. In several embodiments, the histamine receptor activator inhibits the activity and/or expression of the histamine H1 receptor.

In several embodiments, the disclosure provides a transdermal patch for use in the treatment or prevention of diseases associated with the over-expression and/or over-activity of the histamine H1 receptor, such as, for example, one or more of migraine headaches, Parkinson's Disease, Alzheimer's Disease, coronary disease, leukemia, amyotrophic lateral sclerosis, epilepsy, histadelia, histapenia, and/or one or more conditions associated with aging. Similarly, there is provided, in several embodiments, a histamine receptor activator for use in the treatment or prevention of diseases associated with the under-expression and/or under-activity one or more of the histamine H2, H3, and/or H4 receptors, such as, for example, migraine headaches, Parkinson's Disease, Alzheimer's Disease, coronary disease, leukemia, amyotrophic lateral sclerosis, epilepsy, histadelia, histapenia, and/or one or more conditions associated with aging.

In several embodiments, the disclosure includes a histamine receptor activator(s) for use in the treatment or prevention of migraine headaches, Parkinson's Disease, Alzheimer's Disease, coronary disease, leukemia, amyotrophic lateral sclerosis, epilepsy, histadelia, histapenia, and/or one or more conditions associated with aging by combined, sequential, or separate administration with a complete or partial antagonist of a histamine H1 receptor.

In several embodiments, the disclosure includes an activator of the activity and/or expression of histamine receptors H1R, H2R, H3R, H4R for use in the treatment or prevention of migraine headaches, Parkinson's Disease, Alzheimer's Disease, coronary disease, leukemia, amyotrophic lateral sclerosis, epilepsy, ageing, histadelia, histapenia, and/or one or more conditions associated with aging.

In some embodiments, the subject may be a subject in need thereof. In some embodiments, the subject in need thereof may include a subject with a disease or a condition. In some embodiments, the disease or condition may include addiction, allergy, anti-inflammation, cardiovascular disease, a dermatological disease or condition, a metabolic disease or condition, an endocrinology disease or condition, a bone disease or condition, a gastroenterology disease or condition, a hematology disease or condition, a coagulation disease or condition, an immunomodulatory disease or condition, a neurological disease or condition, an oncological disease or condition, a psychiatric disease or condition, a pulmonary disease or condition, a renal disease or condition, a rheumatological disease or condition, or a urological disease or condition.

In some embodiments, the patch is applied to a subject's skin for about 0.5 hours to about 72 hours. In some embodiments, the patch is applied to a subject's skin for about 1 hour, 2 hours, 4 hours, 5 hours, 6 hours, 7 hours, 8 hours, 9 hours, 10 hours, 11 hours, 12 hours, 13 hours, 14 hours, 15 hours, 16 hours 20 hours, 21 hours, 22 hours, 23 hours, 24 hours, 26 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, 72 hours, or ranges including and/or spanning the aforementioned values.

Depending on the embodiment, the duration of each dosing segment can vary. In several embodiments, the duration of the segment ranges from about one day to about one week, including about 1 to about 2 days, about 2 to about 4 days, about 4 days to about 6 days, about 5 days to about 7 days, or ranges including and/or spanning the aforementioned values. In several embodiments, the dosing segments range from about 1 week to about 16 weeks, including about 1 to about 2 weeks, about 2 to about 3 weeks, about 3 to about 4 weeks, about 4 to about 6 weeks, about 6 to about 9 weeks, about 9 to about 12 weeks, about 12 to about 16 weeks, or ranges including and/or spanning the aforementioned values.

In some embodiments, the patch does not substantially irritate the subject's skin. As used herein, the term “does not substantially irritate a subject's skin” includes a resulting skin erythema score of about 3.0 or less, preferably about 2.0 or less, more preferably about 1.0 or less upon patch removal. In another embodiment, the conditions “does not substantially irritate a subject's skin” may refer to the skin irritation relief days of about 4.0 or less, preferably about 3.0 or less, more preferably about 2.5 or less. In another embodiment, the conditions “does not substantially irritate a subject's skin” if the resulting skin erythema score of about 3.0 or less, preferably about 2.0 or less, more preferably about 1.0 or less upon patch removal and the skin irritation relief days of about 4.0 or less, preferably about 3.0 or less, more preferably about 2.5 or less.

In some aspects, the disclosure also includes a system for using a patch to administer an active agent to a subject in need thereof. In some embodiments, the transdermal drug delivery patch system includes applying a transdermal patch to a subject's skin. In some embodiments, the drug delivery patch includes a top layer including an adhesive, a middle layer that has at least one reservoir for containing an active agent, and a bottom layer, wherein the bottom layer includes a release liner. In some embodiments, a reservoir or a reservoir matrix could also contain excipient compounds which enhance the effect of a separately contained bioactive agent. In some embodiments, a reservoir or a reservoir agent could contain or be treated with reactive enzymes or reagents designed to allow the measurement or detection of a selected analyte in an extracted biological fluid. In some embodiments, a reservoir or reservoir matrix may include an open volume space, a gel, a flat planar space which has been coated or treated with a selected compound for subsequent release or reaction, or a permeable solid structure such as a pellet, tablet or porous polymer.

In some aspects, a patch as described herein may be formulated for veterinary application. In some embodiments, the patch includes a veterinary carrier. Veterinary carriers are materials useful for the purpose of administering the composition and may be solid, liquid, or gaseous materials which are otherwise inert or acceptable in the veterinary art and are compatible with the active agent. In some embodiments, the patch for veterinary application may be administered transdermally or by any other desired route. In some embodiments, the formulation as described herein can also include veterinary formulations, e.g., pharmaceutical preparations of the subject formulations suitable for veterinary uses. In some embodiments, a method is provided to administer a patch to an animal. In some embodiments, the patch includes a top layer comprising an adhesive, a middle layer comprising an active agent and a veterinary carrier, wherein the active agent is a histamine or salt thereof; and a bottom layer, wherein the bottom layer comprises a release liner.

In several embodiments, a method for restoring histamine balance is provided. In some embodiments, normalizing activity of histamine receptors is achieved through administration of one or more transdermal patches as described herein. In several embodiments, the methods comprise administering at least a first, a second, and a third initial therapy segments, each of the initial segments comprising activating a histamine H1, H2, H3 or H4 receptor by administering to a subject a transdermal patch, cream, lotion, spray that activates the H1, H2, H3 and H4 receptors. In some embodiments, during the process of histamine receptor activation, additional endogenous histamine is released. In some embodiments, the process results in a dosage higher than the dosage of histamine contained in the patch. Each histamine receptor (H1R, H2R, H3R, H4R) has affinity to a specific histamine concentration. Expression of Histamine Receptors (HRs) in resident and nonresident cells of the skin are described in Table 1.

TABLE 1

| | Histamine Receptor (HR) |
|---|---|
| Cell Type | Expression |
| Mast cells | H1R, H2R, H3R, H4R |
| Eosinophils | H1R, H2R, H3R, H4R |
| Basophils | H4R |
| Neutrophils | H1R, H2R, H4R |
| Dendritic cells | H1R, H2R, H3R, H4R |
| Langrhans cells | H4R |
| Monocytes macrophages | H1R, H2R, H3R, H4R |
| T cells | H1R, H2R, H4R |
| B cells | H1R, H2R |
| Keratinocytes | H1R, H2R |
| Endothelial cells | H1R, H2R, H3R, H4R |
| Smooth muscle cells | H1R, H2R |
| Fibroblasts | H1R |
| Neurons | H1R, H2R, H3R |

For example, if the patch contains 20 ng of histamine dihydrochloride, through the above-mentioned histamine receptor modulation process, the overall histamine generated can be 2000 ng of histamine, some endogenous, some generated directly from the patch. Not wishing to be bound by theory, the affinity for histamine for each histamine receptor is described in Table 2.

TABLE 2

| Histamine Receptor | Affinity for Histamine | |
|---|---|---|
| H1R | ~1.1 | μg/ml |
| H2R | ~3.3 | μg/ml |
| H3R | ~1.1 | ng/ml |
| H4R | ~2.2-4.4 | ng/ml |

Accordingly, some aspects described herein relate to the following numbered alternatives:

1. A patch for delivering an active agent to a subject in need thereof, comprising: a top layer comprising an adhesive, the adhesive containing the active agent; a middle layer comprising an active agent, wherein the active agent is a histamine or salt thereof; and a bottom layer, wherein the bottom layer comprises a release liner.

2. The patch of alternative 1, wherein the active agent is 0.001 μg/mL to about 30 μg/mL histamine or salt thereof.

3. The patch of alternative 1 or 2, wherein the middle layer further comprises a carrier.

4. The patch of alternative 3, wherein the carrier comprises water, saline, saccharides, polysaccharides, buffers, excipients, biodegradable polymers, liposomes, stabilizer, skin irritation reducer, and mixtures thereof.

5. The patch of any one of alternative 1 to 4, wherein the middle layer is a gel.

6. The patch of alternative 5, wherein the gel is a hydrogel.

7. The patch of any one of alternatives 1 to 6, wherein the release liner comprises a polyester liner or a polyethylene liner.

8. The patch of any one of alternatives 1 to 7, wherein the top liner comprises a polyester liner or a polyethylene liner.

9. The patch of any one of alternatives 1 to 8, wherein the patch is at least 0.5 cm by 0.5 cm.

10. The patch of any one of alternatives 1 to 9, wherein the patch is at least 0.01 mm thick to about 2 mm thick.

11. The patch of any one of alternatives 1 to 10, wherein the middle layer further comprises a permeation enhancing agent.

12. The patch of any one of alternatives 1 to 11, wherein the middle layer further comprise an absorption enhancer.

13. The patch of any one of alternatives 1 to 12, wherein the patch has a pH from about 1.5 to about 8.

14. The patch of any one of alternatives 1 to 13, wherein the patch is a transdermal patch.

15. The patch of alternative 14, wherein the transdermal patch is selected from the group consisting of Single-layer Drug-in-Adhesive, Multi-layer Drug-in-Adhesive, Reservoir Matrix, Vapour Patch, and Microneedle patch.

16. A method of modulating histamine in a subject, the method comprising administering to the subject the patch according to any one of alternatives 1 to 15.

17. The method of alternative 16, wherein the subject's plasma histamine levels are lowered.

18. The method of alternative 16 or 17, wherein the subject's blood histamine concentration is lowered.

19. The method of any one of alternatives 16 to 18, wherein the serum histamine level in the subject are lowered by at least 10 ng/ml.

20. The method of any one of alternatives 16 to 18, wherein the serum histamine levels in the subject are reduced from about 100 ng/ml to about 45 ng/ml.

21. The method of any one of alternatives 16 to 20, wherein the patch is applied to the subjects skin from about 0.5 hours to about 72 hours.

22. The method of any one of alternatives 16 to 21, wherein more than one patch is applied to the subject's skin.

23. The method of any one of alternatives 16 to 21, wherein a subsequent patch is applied to a subject's skin after 0.5 to about 72 hours after the first patch is applied to the subject's skin.

24. The method of any one of alternatives 16 to 22, wherein one or more patch is applied to the subject's skin over a 14 day treatment cycle up to 1 year treatment applied at varying time intervals.

25. A method of ameliorating, reducing, or treating a migraine headache of a subject, the method comprising administering to the subject the patch according to any one of alternatives 1 to 15.

26. The method of alternative 25, wherein the subject's plasma histamine levels are lowered.

27. The method of alternative 25 or 26, wherein the subject's blood histamine concentration is lowered.

28. The method of any one of alternatives 25 to 27, wherein the serum histamine level in the subject are lowered by at least 10 ng/ml.

29. The method of any one of alternatives 25 to 28, wherein the serum histamine levels in the subject are reduced from about 100 ng/ml to about 45 ng/ml.

30. The method of any one of alternatives 25 to 29, wherein the patch is applied to the subjects skin from about 0.5 hours to about 72 hours.

31. The method of any one of alternatives 25 to 30, wherein more than one patch is applied to the subject's skin.

32. The method of any one of alternatives 25 to 31, wherein a subsequent patch is applied to a subject's skin after 0.5 to about 72 hours after the first patch is applied to the subject's skin.

33. The method of any one of alternatives 25 to 32, wherein one or more patch is applied to the subject's skin over a 14 day treatment cycle up to 1 year treatment applied at varying time intervals.

34. The method of anyone of alternatives 25 to 33, wherein the histamine levels during a migraine event are lowered.

35. The method of anyone of alternatives 25 to 34, wherein the histamine levels during a symptom free state is lowered.

36. A method of modulating brain histamine levels in a subject with Parkinson's disease, the method comprising administering to the subject a patch according to any one of alternatives 1 to 15.

37. The method of alternative 36, wherein the subject's plasma histamine levels are lowered.

38. The method of alternative 36 or 37, wherein the subject's blood histamine concentration is lowered.

39. The method of any one of alternatives 38 to 38, wherein the serum histamine level in the subject are lowered by at least 10 ng/ml.

40. The method of any one of alternatives 36 to 39, wherein the serum histamine levels in the subject are reduced from about 100 ng/ml to about 45 ng/ml.

41. The method of any one of alternatives 36 to 40, wherein the patch is applied to the subject's skin from about 0.5 hours to about 72 hours.

42. The method of any one of alternatives 36 to 41, wherein more than one patch is applied to the subject's skin.

43. The method of any one of alternatives 36 to 42, wherein a subsequent patch is applied to a subject's skin after 0.5 to about 72 hours after the first patch is applied to the subject's skin.

44. The method of any one of alternatives 36 to 43, wherein one or more patch is applied to the subject's skin over a 14 day treatment cycle up to 1 year treatment applied at varying time intervals.

45. A method of modulating brain histamine levels in a subject with Alzheimer's disease, the method comprising administering to the subject the patch according to any one of alternatives 1 to 15.

46. The method of alternative 45, wherein the subject's plasma histamine levels are lowered.

47. The method of alternative 45 or 46, wherein the subject's blood histamine concentration is lowered.

48. The method of any one of alternatives 45 to 47, wherein the serum histamine level in the subject are lowered by at least 10 ng/ml.

49. The method of any one of alternatives 45 to 48, wherein the serum histamine levels in the subject are reduced from about 100 ng/ml to about 45 ng/ml.

50. The method of any one of alternatives 45 to 49, wherein the patch is applied to the subjects skin from about 0.5 hours to about 72 hours.

51. The method of any one of alternatives 45 to 50, wherein more than one patch is applied to the subject's skin.

52. The method of any one of alternatives 45 to 51, wherein a subsequent patch is applied to a subject's skin after 0.5 to about 72 hours after the first patch is applied to the subject's skin.

53. The method of any one of alternatives 45 to 52, wherein one or more patch is applied to the subject's skin over a 14 day treatment cycle up to 1 year treatment applied at varying time intervals.

54. A method of treating a subject with elevated histamine levels, the method comprising administering to the subject a first dosing segment comprising the patch according to any one of alternatives 1 to 15 and administering to the subject a second dosing segment comprising the patch according to any one of alternatives 1 to 15.

55. The method of alternative 54, further comprising administering to the subject a third dosing segment comprising the patch according to any one of alternatives 1 to 15.

56. The method of alternative 54 or 55, further comprising administering to the subject a forth dosing segment comprising the patch according to any one of alternatives 1 to 15.

57. The method of any one of alternatives 54 to 56, wherein each patch from the first dosing segment and the second dosing segment comprises a different concentration of the active agent.

58. A transdermal drug delivery system for delivering at least one drug into a tissue membrane of a subject, the system comprising a) an applicator configured to supply a drug delivery patch and b) a patch, wherein the patch comprises a top layer comprising an adhesive, a middle layer comprising at least one drug, and a bottom layer, wherein the bottom layer comprises a release liner, wherein the at least one drug is a histamine receptor modulator.

59. The transdermal drug delivery system of alternative 58, wherein the histamine receptor modulator activates histamine H1 receptors.

60. The transdermal drug delivery system of alternative 58 or 59, wherein the histamine receptor modulator activates histamine H2 receptors.

61. The transdermal drug delivery system of any one of alternatives 58 to 60, wherein the histamine receptor modulator activates histamine H3 receptors.

62. The transdermal drug delivery system of any one of alternatives 58 to 61, wherein the histamine receptor modulator activates histamine H4 receptors.

63. The transdermal drug delivery system of any one of alternatives 58 to 62, wherein the histamine receptor modulator is histamine, a histamine salt, or a combination thereof.

64. The transdermal drug delivery system of alternative 63, wherein the histamine salt is selected from the group consisting of histamine diphosphate, N-alpha methyl histamine, histamine phosphate, histamine dihydrochloride, or a combination thereof.

65. The transdermal drug delivery system of alternative 58, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof.

66. The transdermal drug delivery system of any one of alternatives 58 to 65, wherein the histamine receptor modulator concentration is 0.01 μg/mL to about 100 μg/mL.

67. The transdermal drug delivery system of any one of alternatives 58 to 66, wherein the middle layer further comprises a carrier.

68. The transdermal drug delivery system of alternative 67, wherein the carrier comprises water, saline, saccharides, polysaccharides, buffers, excipients, biodegradable polymers, liposomes, stabilizer, skin irritation reducer, and mixtures thereof.

69. The transdermal drug delivery system of any one of alternatives 58 to 68, wherein the middle layer is a gel.

70. The transdermal drug delivery system of alternative 69, wherein the gel is a hydrogel.

71. The transdermal drug delivery system of any one of alternatives 58 to 70, wherein the release liner comprises a polyester liner or a polyethylene liner.

72. The transdermal drug delivery system of any one of alternatives 58 to 71, wherein the top liner comprises a polyester liner or a polyethylene liner.

73. The transdermal drug delivery system of any one of alternatives 58 to 72, wherein the patch is at least 1 cm by 1 cm.

74. The transdermal drug delivery system of any one of alternatives 58 to 73, wherein the patch is at least 0.01 mm thick to about 2 mm thick.

75. The transdermal drug delivery system of any one of alternatives 58 to 74, wherein the middle layer further comprises a permeation-enhancing agent.

76. The transdermal drug delivery system of any one of alternatives 58 to 75, wherein the middle layer further comprises an absorption enhancer.

77. The transdermal drug delivery system of any one of alternatives 58 to 76, wherein the patch has a pH from about 3 to about 8.

78. The transdermal drug delivery system of any one of alternatives 58 to 77, wherein the applicator is configured for microporation delivery of the patch.

79. The transdermal drug delivery system of any one of alternatives 58 to 78, wherein the applicator is configured for microneedle delivery of the patch.

80. The transdermal drug delivery system of any one of alternatives 58 to 79, wherein the patch is selected from the group consisting of single-layer drug-in-adhesive, multi-layer drug-in-adhesive, reservoir matrix, vapor patch, and microneedle.

81. A transdermal drug delivery system for delivering at least one drug into a tissue membrane of a subject, the system comprising a patch, wherein the patch comprises a top layer comprising an adhesive, a middle layer comprising at least one drug, and a bottom layer, wherein the bottom layer comprises a release liner, wherein the at least one drug is a histamine receptor modulator.

82. The transdermal drug delivery system of alternative 81, wherein the histamine receptor modulator activates histamine H1 receptors.

83. The transdermal drug delivery system of alternative 81 or 82, wherein the histamine receptor modulator activates histamine H2 receptors.

84. The transdermal drug delivery system of any one of alternatives 81 to 83, wherein the histamine receptor modulator activates histamine H3 receptors.

85. The transdermal drug delivery system of any one of alternatives 81 to 84, wherein the histamine receptor modulator activates histamine H4 receptors.

86. The transdermal drug delivery system of any one of alternatives 81 to 85, wherein the histamine receptor modulator is histamine, a histamine salt, or a combination thereof.

87. The transdermal drug delivery system of alternative 86, wherein the histamine salt is selected from the group consisting of histamine diphosphate, N-alpha methyl histamine, histamine phosphate, histamine dihydrochloride, or a combination thereof.

88. The transdermal drug delivery system of alternative 81, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof.

89. The transdermal drug delivery system of any one of alternatives 81 to 88, wherein the histamine receptor modulator concentration is 0.001 μg/mL to about 100 μg/mL.

90. The transdermal drug delivery system of any one of alternatives 81 to 89, wherein the middle layer further comprises a carrier.

91. The transdermal drug delivery system of alternative 90, wherein the carrier comprises water, saline, saccharides, polysaccharides, buffers, excipients, biodegradable polymers, liposomes, stabilizer, skin irritation reducer, and mixtures thereof.

92. The transdermal drug delivery system of any one of alternatives 81 to 91, wherein the middle layer is a gel or pressure sensitive adhesive.

93. The transdermal drug delivery system of alternative 92, wherein the gel is a hydrogel or pressure sensitive adhesive.

94. The transdermal drug delivery system of any one of alternatives 81 to 93, wherein the release liner comprises a polyester liner or a polyethylene liner.

95. The transdermal drug delivery system of any one of alternatives 81 to 94, wherein the top liner comprises a polyester liner or a polyethylene liner.

96. The transdermal drug delivery system of any one of alternatives 81 to 95, wherein the patch is at least 0.5 cm by 0.5 cm.

97. The transdermal drug delivery system of any one of alternatives 81 to 96, wherein the patch is at least 0.01 mm thick to about 2 mm thick.

98. The transdermal drug delivery system of any one of alternatives 81 to 97, wherein the middle layer further comprises a permeation-enhancing agent.

99. The transdermal drug delivery system of any one of alternatives 81 to 98, wherein the middle layer further comprises an absorption enhancer.

100. The transdermal drug delivery system of any one of alternatives 81 to 99, wherein the patch has a pH from about 3 to about 8.

101. The transdermal drug delivery system of alternative 100, wherein the patch further comprises a rate controlling membrane.

102. The transdermal drug delivery system of any one of alternatives 81 to 101, wherein the adhesive is a pressure-sensitive adhesive.

103. The transdermal drug delivery system of any one of alternatives 81 to 102, wherein the top layer comprises a non-woven backing, polyethylene backing or any other standard backing.

104. The transdermal drug delivery system of any one of alternatives 81 to 103, wherein the patch is selected from the group consisting of single-layer drug-in-adhesive, multi-layer drug-in-adhesive, reservoir matrix, vapor patch, and microneedle.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

The model developed allows for correlating the concentration of the Franz Cell receiving solution or PermeGear InLine Cells (ILC07) (In-Line Cells—PermeGear) (INPUT) to the concentration of the delivered drug (histamine receptor modulator) in the viable epidermis (OUTPUT) for a given trans-dermal patch location. For example, locating the transdermal patch on the shoulder, with a viable epidermis thickness of 70.3 μm will result in the following Franz cell receptor solution histamine concentration to viable epidermis histamine concentration correlation:

TABLE 3

| | Stratum | Viable | Total |
|---|---|---|---|
| Parameter | corneum (μm) | epidermis (μm) | epidermis (μm) |
| Forearm dorsal | 18.3 (4.9) | 56.6 (11.5) | 74.9 |
| Shoulder | 11.0 (2.2) | 70.3 (13.6) | 81.3 |
| Buttock | 14.9 (3.4) | 81.5 (15.7) | 96.5 |
| All body Sites | 14.8 (4.8) | 69.9 (17.0) | 83.7 |

The thickness of stratum corneum and viable epidermis at different body sites-mean (standard deviation). The data was taken from an evaluated 71 subjects (37 males, 34 females, age: 20-68 years, median: 47 years) and reported the data on each patient.

Patients experiencing a number of conditions have increased levels of histamine compared to the healthy population. Through the designed drug release profile, the trans-dermal device will modulate the histamine receptors involved in these indications and over time it will reduce the histamine levels to the levels of the healthy subjects. Trans-dermal device with a drug release profile in the Nano-grams/mL concentration range will particularly modulate H3R and H4R histamine receptors while the drug release profile in the micro-grams/mL concentration range will particularly modulate H1R and H2R histamine receptors.

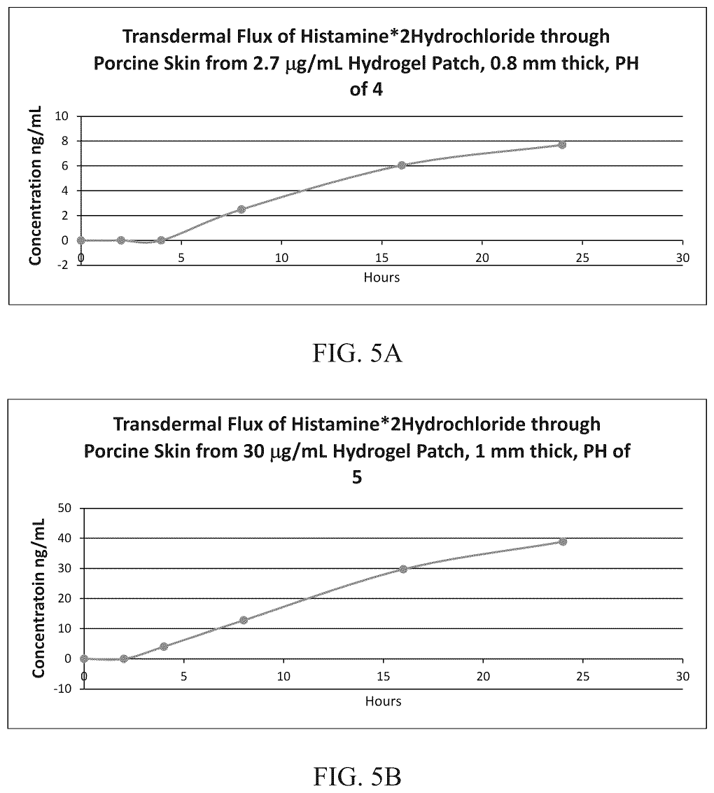

As the concentration of histamine varies through the skin, at various skin thickness (epidermis, dermis, hypodermis), specific histamine receptors will be modulated/activate resulting in a biological response (see FIG. 5A). As the concentration of histamine varies through the skin, at various skin thickness (epidermis, dermis, hypodermis), specific histamine receptors will be modulated/activated resulting in a biological response (FIG. 5B).

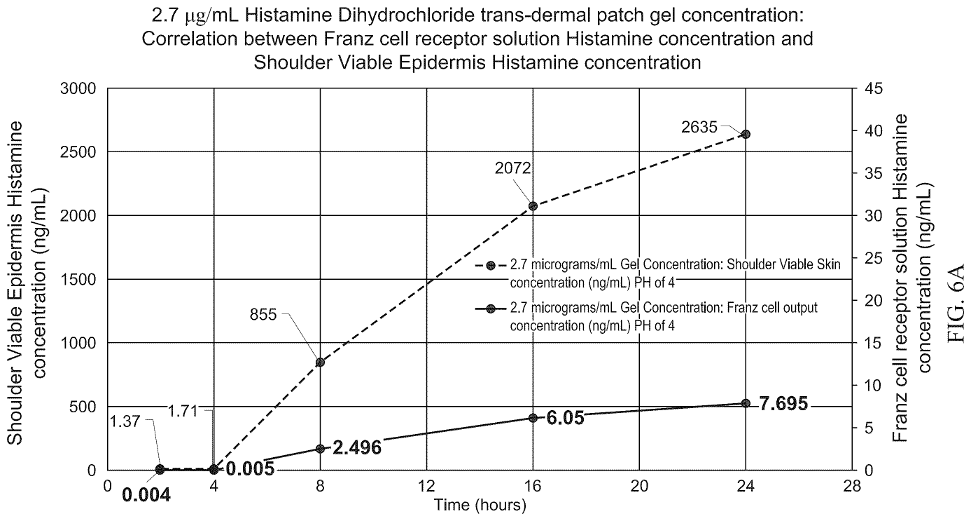

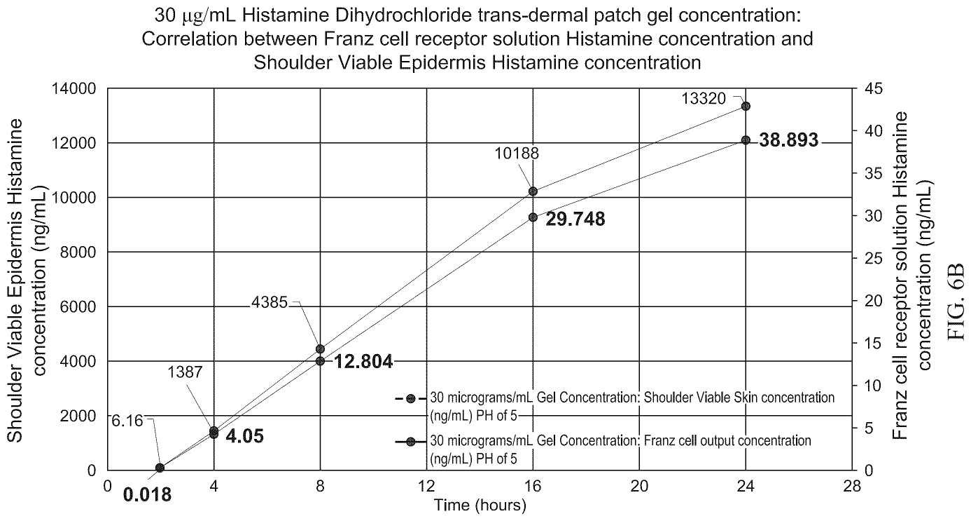

As the concentration of histamine varies through the skin, at various skin thickness (epidermis, dermis, hypodermis), specific histamine receptors (H1R, H2R, H3R, H4R) will be modulated/activate resulting in a biological response (see FIG. 6A). The activation of these histamine receptors, over time, will result in balanced histamine levels in the body, preventing migraine/disease from occurring. Disparate affinities of histamine for its receptors allows for preferential activation. Histamine H2 receptors (H2R) inhibit mast cell and basophil activation by elevating cyclic AMP. Histamine H3 receptors (H3R) mediate histamine synthesis and inhibit histamine release. Histamine H4 receptors (H4R) mediate the chemotaxis of mast cells. Histamine H4 receptor (H4R) has the potential of reducing cortical hyper-excitability which is a fundamental feature in the migrainous brain. As the concentration of histamine varies through the skin, at various skin thickness (epidermis, dermis, hypodermis), specific histamine receptors (H1R, H2R, H3R, H4R) will be modulated/activate resulting in a biological response (see FIG. 6B). The activation of these histamine receptors, over time, will result in balanced histamine levels in the body, preventing migraine/disease from occurring. Disparate affinities of histamine for its receptors allows for preferential activation. Histamine H2 receptors (H2R) inhibit mast cell and basophil activation by elevating cyclic AMP. Histamine H3 receptors (H3R) mediate histamine synthesis and inhibit histamine release. Histamine H4 receptors (H4R) mediate the chemotaxis of mast cells. Histamine H4 receptor (H4R) has the potential of reducing cortical hyper-excitability which is a fundamental feature in the migrainous brain.

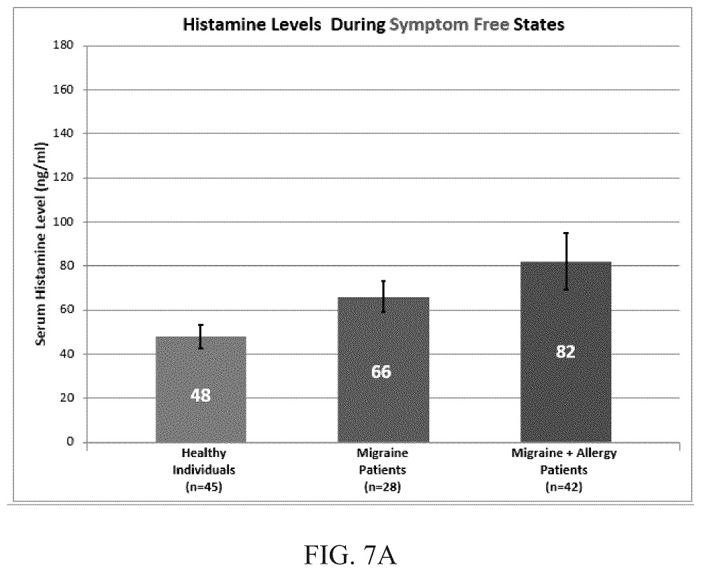

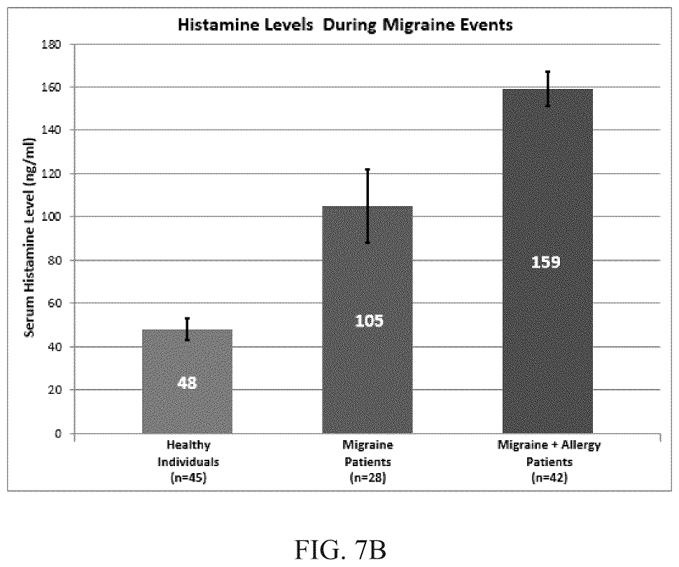

As noted from the graph of FIG. 7A, the histamine levels in healthy individuals are lower (statistically significant) than the histamine levels of people suffering from migraine (during symptom free state) which are lower than the histamine levels of people suffering from both migraine and allergies (during symptom free state). As noted from the graph of FIG. 7B, the histamine levels in healthy individuals are lower (statistically significant) than the histamine levels of people suffering from migraine (during a migraine event) which are lower than the histamine levels of people suffering from both migraine and allergies (during a migraine event). Formulation and dosage forms of the transdermal patch, cream, lotion, spray, and pill as described herein are expected to modulate specific histamine receptors in ways that stabilize mast cell membranes (up-stream), thereby decreasing mast cell degranulation in susceptible migraine patients, preventing migraine initiation. and (over time) reducing histamine levels in migraine patients to levels of healthy individuals.

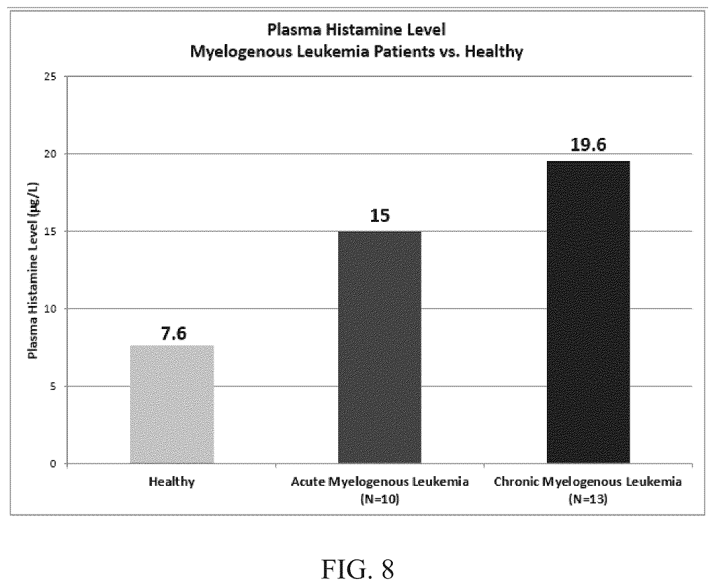

As noted from the graph of FIG. 8, the histamine levels in healthy individuals are lower (statistically significant) than the histamine levels of people suffering form leukemia. Formulation and dosage forms of the transdermal patch, cream, lotion, spray, and pill as described herein are expected to modulate specific histamine receptors in ways that stabilize mast cell membranes (up-stream), thereby decreasing mast cell degranulation in susceptible leukemia patients, treating and preventing leukemia and (over time) reducing histamine levels in leukemia patients to levels of healthy individuals.

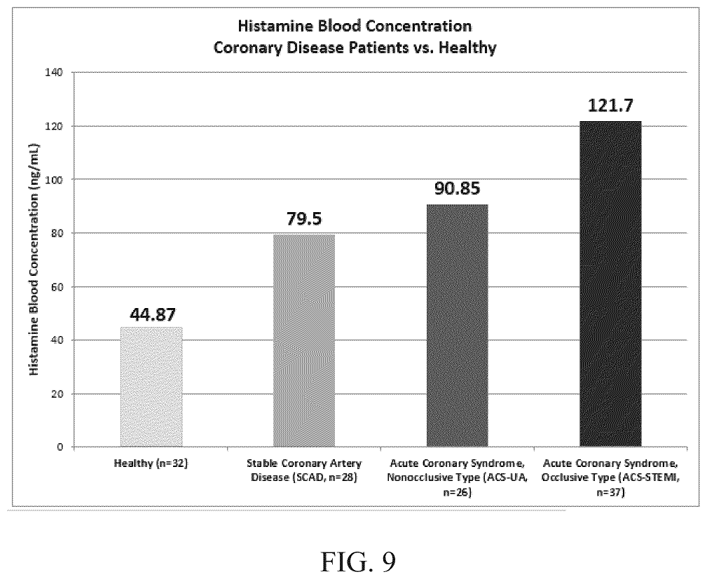

As noted from the graph of FIG. 9, the histamine levels in healthy individuals are lower (statistically significant) than the histamine levels of people suffering from coronary disease. Formulation and dosage forms of the transdermal patch, cream, lotion, spray, and pill as described herein are expected to modulate specific histamine receptors in ways that stabilize mast cell membranes (up-stream), thereby decreasing mast cell degranulation in susceptible coronary disease patients, treating and preventing coronary disease and (over time) reducing histamine levels in coronary disease patients to levels of healthy individuals.

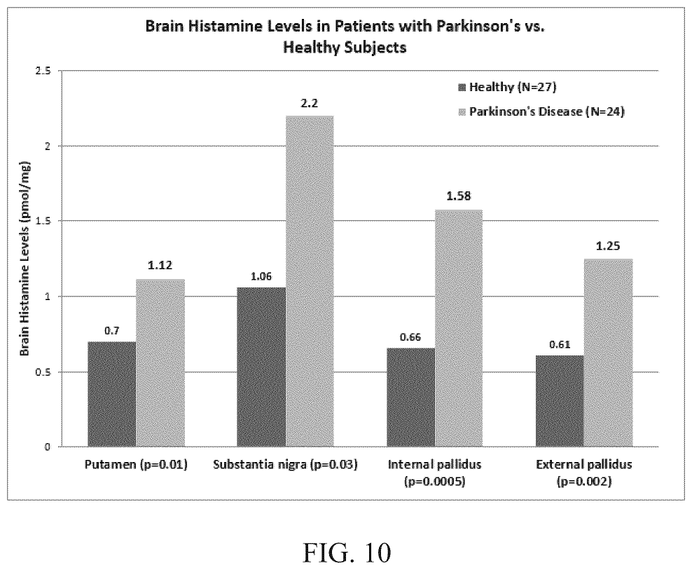

As noted from the graph of FIG. 10, the histamine levels in healthy individuals are lower (statistically significant) than the histamine levels of people suffering from Parkinson's disease. Formulation and dosage forms of the transdermal patch, cream, lotion, spray, and pill as described herein are expected to modulate specific histamine receptors in ways that stabilize mast cell membranes (up-stream), thereby decreasing mast cell degranulation in susceptible Parkinson's disease patients, treating and preventing Parkinson's disease and (over time) reducing histamine levels in Parkinson's disease patients to levels of healthy individuals.

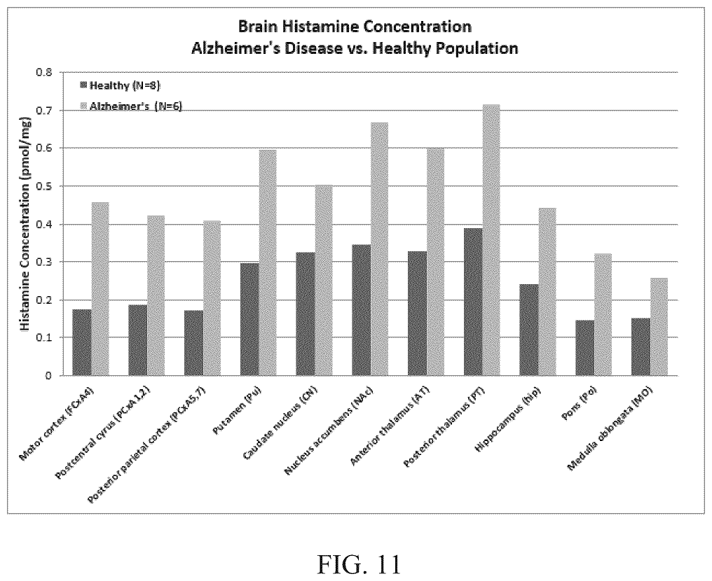

As noted from the graph of FIG. 11, the histamine levels in healthy individuals are lower (statistically significant) than the histamine levels of people suffering from Alzheimer's disease. Formulation and dosage forms of the transdermal patch, cream, lotion, spray, and pill as described herein are expected to modulate specific histamine receptors in ways that stabilize mast cell membranes (up-stream), thereby decreasing mast cell degranulation in susceptible Alzheimer's disease patients, treating and preventing Alzheimer's disease and (over time) reducing histamine levels in Alzheimer's disease patients to levels of healthy individuals.

### Example 2

A subject in need of histamine modulating is provided a transdermal patch comprising 2.7 μg/mL histamine dihydrochloride to the subject's skin. The patch is applied to the subject's skin for 24 hours. The histamine dihydrochloride present in the patch is transferred by diffusion from the patch through the subject's skin. The size of the patch can vary from ¼ inch by ¼ inch to 3 inches by 3 inches depending on the desired dosage to be administered.

### Example 3

A subject with a history of migraine headaches is provided a transdermal patch comprising 30 μg/mL histamine dihydrochloride to the subject's skin during a migraine event. The patch is applied to the subject's skin for 4 hours. The histamine dihydrochloride present in the patch is transferred by diffusion from the patch through the subject's skin. As a result, one or more migraine events are treated or reduced. The size of the patch can vary from ¼ inch by ¼ inch to 1 inch by 1 inch depending on the desired dosage to be administered.

### Example 4

A male subject suffering from Alzheimer's disease is treated with a transdermal patch as described herein. The active agent comprised 10 μg/mL histamine dihydrochloride and was applied to the subject's skin every day for two hours a day. The treatment regimen occurred over the course of a month. As a result, one or more Alzheimer's disease symptoms was treated or reduced.

### Example 5

A subject suffering from migraine headaches is administered a 2″×2″ patch with a gel concentration of 30 μg/ml over a 24 hour period. The approximate dose of histamine administered was 2,600 ng. As a result, the subject's migraine headaches were reduced in both intensity and duration.

### Example 6

A subject suffering from migraine headaches is administered a 2″×2″ patch with a gel concentration of 2.7 μg/ml over a 24 hour period. The approximate dose of histamine administered was 500 ng. As a result, the subject's migraine headaches were reduced in both intensity and duration. In addition to the histamine dihydrochloride contained in the patch, through the activation of H1R-H4R histamine receptor, additional histamine is released from histamine containing cells (mast cells, basophils, etc.).

### Example 7

A pressure sensitive patch is prepared utilizing an acrylic based adhesive which additionally contains oleic acid, dipropylene glycol and sodium polyacrylate with a polyester backing and a polyester release liner. The pressure sensitive patch is administered as a 1″×1″ patch with a concentration of histamine dihydrochloride of 2 or 3 μg/ml.

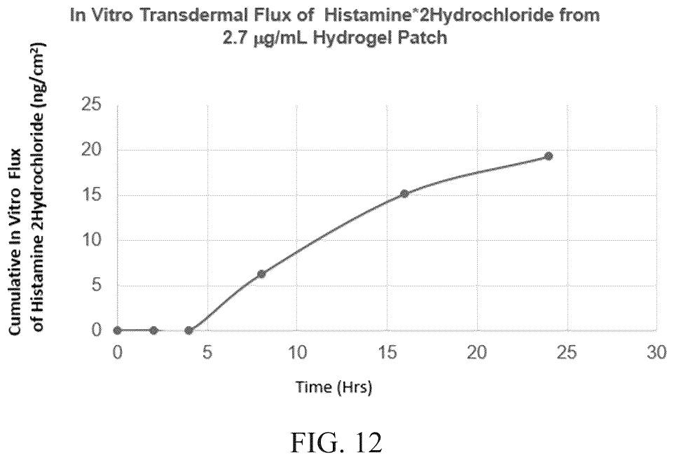

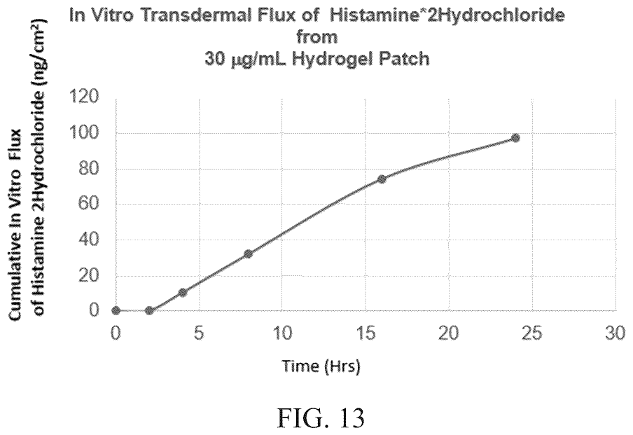

The histamine release profile can be further modified by altering the concentration of histamine dihydrochloride (histamine/histamine salt/histamine receptor modulator) and adding/subtracting “penetration enhancer” depending on which receptors are targeted (see FIGS. 12 and 13).

### Example 8

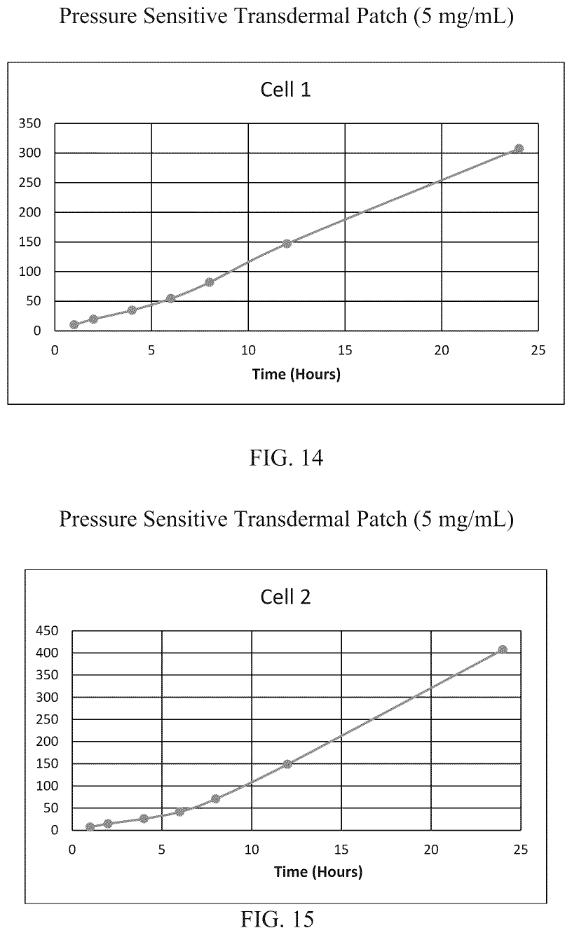

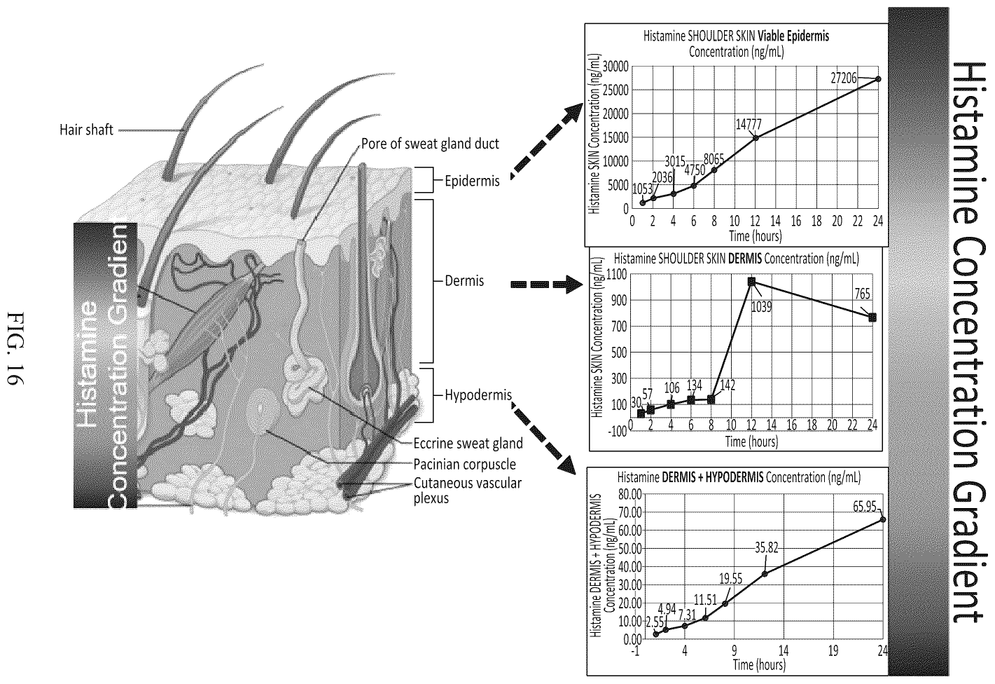

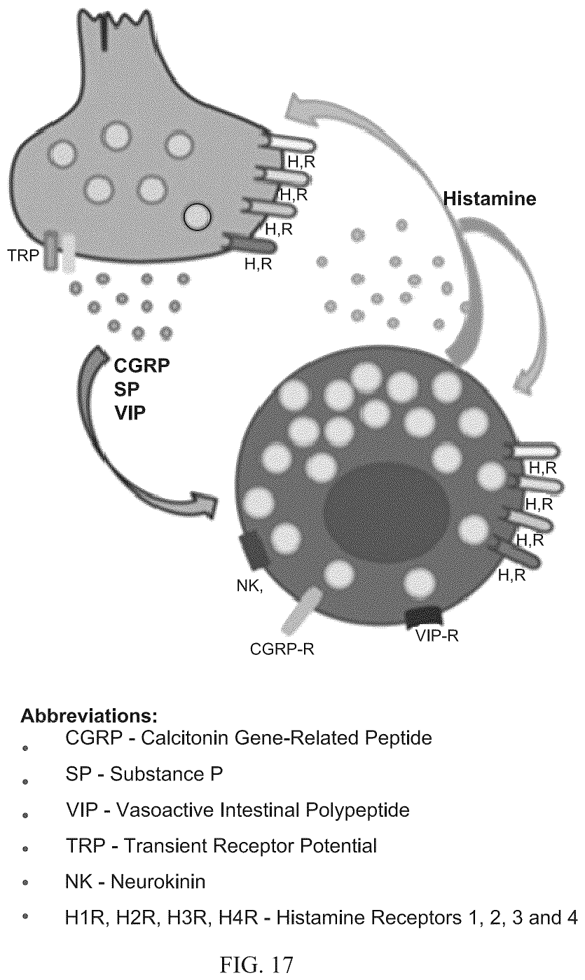

In this experiment, two runs were performed with five formulations utilizing 14 in-line cells. For this experiment, the porcine skin was cut into 2.0 cm×2.0 cm squares and overlayed on receptor fluid in a common vessel for 48 hours. The skin was installed in sections in the In-Line cells and receptor fluid was pumped through the cells at 0.5 mL/hour for 12 hours prior to the application of formulations. The effluent was collected in tared 20 mL vials. The patch formulations was cut into 1.77 cm² discs. The pump was stopped, removed and the vials were weighed. The donor cover was carefully removed from each cell and the patch formulations were applied to the center of the skin sections. The donor cover was replaced and the cells were clamped. The vial racks were labeled with tared 2 ml vials. The fraction collector was programed to move the racks at the defined sample times. The pump was restarted at 0.5 mL/hour. At the completion of the test, the vials were weighed. Each vial was labeled and capped. The net weights were calculated and recorded as volumes in the prepared spreadsheet. The samples were ice-packed and further analyzed (see FIGS. 12-16).

What is claimed is:

1. A patch for delivering an active agent to a subject in need thereof, comprising:
a top layer comprising an adhesive, the adhesive containing the active agent;
a middle layer comprising an active agent, wherein the active agent is a histamine or salt thereof; and
a bottom layer, wherein the bottom layer comprises a release liner.

2. The patch of claim 1, wherein the active agent is 0.001 μg/mL to about 100 μg/mL histamine or salt thereof.

3. The patch of claim 1, wherein the middle layer further comprises a carrier.

4. The patch of claim 3, wherein the carrier comprises water, saline, saccharides, polysaccharides, buffers, excipients, biodegradable polymers, liposomes, stabilizer, skin irritation reducer, and mixtures thereof.

5. The patch of any one of claims 1 to 4, wherein the middle layer is a gel.

6. The patch of claim 5, wherein the gel is a hydrogel.

7. The patch of any one of claims 1 to 4, wherein the release liner and top liner comprises a polyester liner or a polyethylene liner.

8. The patch of any one of claims 1 to 4, wherein the patch is at least 0.01 mm thick to about 2 mm thick.

9. The patch of any one of claims 1 to 4, wherein the middle layer further comprises a permeation enhancing agent.

10. The patch of any one of claims 1 to 4, wherein the middle layer further comprise an absorption enhancer.

11. The patch of any one of claims 1 to 4, wherein the patch has a pH from about 1.5 to about 8.

12. The patch of any one of claims 1 to 4, wherein the patch is a transdermal patch selected from the group consisting of a single-layer drug-in-adhesive patch, multi-layer drug-in-adhesive patch, reservoir matrix, vapour patch, and microneedle patch.

13. A method of modulating histamine in a subject, the method comprising:
administering to the subject the patch according to any one of claims 1 to 12.

14. The method of claim 13, wherein the subject's plasma, serum, buffy coat, whole blood histamine levels are lowered.

15. The method of claim 13, wherein the subject's blood/serum/plasma/buffy coat histamine concentration is lowered.

16. The method of any one of claims 13 to 15, wherein the serum/plasma/buffy coat histamine levels in the subject are reduced from about 100 ng/ml to about 45 ng/ml.

17. The method of any one of claims 13 to 15, wherein a subsequent patch is applied to a subject's skin after 0.5 to about 72 hours after the first patch is applied to the subject's skin.

18. The method of any one of claims 13 to 15, wherein one or more patch is applied to the subject's skin over a 14 day treatment cycle up to 1 year treatment applied at varying time intervals.

19. A method of treating a subject with elevated histamine levels, the method comprising:
administering to the subject a first dosing segment comprising the patch according to any one of claims 1 to 12; and
administering to the subject a second dosing segment comprising the patch according to any one of claims 1 to 12.

20. The method of claim 19, further comprising administering to the subject a third dosing segment comprising the patch according to any one of claims 1 to 12.

21. The method of claim 19 or 20, further comprising administering to the subject a forth dosing segment comprising the patch according to any one of claims 1 to 12.

22. The method of any one of claims 19 to 21, wherein each patch from the first dosing segment and the second dosing segment comprises a different concentration of the active agent.

23. A transdermal drug delivery system for delivering at least one drug into a tissue membrane of a subject, the system comprising:
a) an applicator configured to supply a drug delivery patch; and
b) a patch, wherein the patch comprises:
a top layer comprising an adhesive,
a middle layer comprising at least one drug, and
a bottom layer, wherein the bottom layer comprises a release liner,
wherein the at least one drug is a histamine receptor modulator.

24. The transdermal drug delivery system of claim 23, wherein the histamine receptor modulator activates histamine H1 receptors.

25. The transdermal drug delivery system of claim 23, wherein the histamine receptor modulator activates histamine H2 receptors.

26. The transdermal drug delivery system of claim 23, wherein the histamine receptor modulator activates histamine H3 receptors.

27. The transdermal drug delivery system of claim 23, wherein the histamine receptor modulator activates histamine H4 receptors.

28. The transdermal drug delivery system of any one of claims 23 to 27, wherein the histamine receptor modulator is histamine, a histamine salt, or a combination thereof.

29. The transdermal drug delivery system of claim 28, wherein the histamine salt is selected from the group consisting of histamine diphosphate, N-alpha methyl histamine, histamine phosphate, histamine dihydrochloride, or a combination thereof.

30. The transdermal drug delivery system of claim 23, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof.

31. The transdermal drug delivery system of any one of claims 23 to 27, wherein the a histamine receptor modulator concentration is 0.01 μg/mL to about 100 μg/mL.

32. The transdermal drug delivery system of any one of claims 23 to 27, wherein the middle layer further comprises a carrier.

33. The transdermal drug delivery system of claim 32, wherein the carrier comprises water, saline, saccharides, polysaccharides, buffers, excipients, biodegradable polymers, liposomes, stabilizer, skin irritation reducer, and mixtures thereof.

34. The transdermal drug delivery system of any one of claims 23 to 27, wherein the middle layer is a gel.

35. The transdermal drug delivery system of claim 34, wherein the gel is a hydrogel.

36. The transdermal drug delivery system of any one of claims 23 to 27, wherein the release liner and top liner comprises a polyester liner or a polyethylene liner.

37. The transdermal drug delivery system of any one of claims 23 to 27, wherein the patch is at least 0.01 mm thick to about 2 mm thick.

38. The transdermal drug delivery system of any one of claims 23 to 27, wherein the middle layer further comprises a permeation-enhancing agent.

39. The transdermal drug delivery system of any one of claims 23 to 27, wherein the middle layer further comprise an absorption enhancer.

40. The transdermal drug delivery system of any one of claims 23 to 27, wherein the patch has a pH from about 3 to about 8.

41. The transdermal drug delivery system of any one of claims 23 to 27, wherein the applicator is configured for microporation delivery of the patch.

42. The transdermal drug delivery system of any one of claims 23 to 27, wherein the applicator is configured for microneedle delivery of the patch.

43. The transdermal drug delivery system of any one of claims 23 to 27, wherein the patch is selected from the group consisting of single-layer drug-in-adhesive, multi-layer drug-in-adhesive, reservoir matrix, vapor patch, and microneedle.

44. A transdermal drug delivery system for delivering at least one drug into a tissue membrane of a subject, the system comprising:
a patch, wherein the patch comprises:
a top layer comprising an adhesive,
a middle layer comprising at least one drug, and
a bottom layer, wherein the bottom layer comprises a release liner,
wherein the at least one drug is a histamine receptor modulator.

45. The transdermal drug delivery system of claim 44, wherein the histamine receptor modulator activates one or more histamine receptor.

46. The transdermal drug delivery system of claim 44, wherein the histamine receptor modulator is histamine, a histamine salt, or a combination thereof.

47. The transdermal drug delivery system of claim 46, wherein the histamine salt is selected from the group consisting of histamine diphosphate, N-alpha methyl histamine, histamine phosphate, histamine dihydrochloride, or a combination thereof.

48. The transdermal drug delivery system of claim 43, the histamine receptor modulator is selected from the group consisting of diphenhydramine, loratadine, cetirizine, fexofenadine, clemastine, rupatadine, ranitidine, cimetidine, famotidine, nizatidine, ABT-239, ciproxifan, clobenpropit, thioperamide, JNJ 7777120, JNJ 10191584, or combinations thereof.

49. The transdermal drug delivery system of any one of claims 44 to 48, wherein the a histamine receptor modulator concentration is 0.001 μg/mL to about 100 μg/mL.

50. The transdermal drug delivery system of any one of claims 44 to 48, wherein the patch is selected from the group consisting of single-layer drug-in-adhesive, multi-layer drug-in-adhesive, reservoir matrix, vapor patch, and microneedle.
